SEQUENCE LISTING (62 sequences)

SEQ ID NO: 1 - protein, 36 aa, Human immunodeficiency virus type 1
YTSLIHSLIEESQNQQEKNEQELLELDKWASLWNWF

SEQ ID NO: 2 - protein, 38 aa, Human immunodeficiency virus type 1
NNLLRAIEAQQHLLQLTVWQIKQLQARILAVERYLKDQ

SEQ ID NO: 3 - protein, 36 aa, Human immunodeficiency virus type 1
YTNTIYTLLEESQNQQEKNEQELLELDKWASLWNWF

SEQ ID NO: 4 - protein, 36 aa, Human immunodeficiency virus type 1
YTGIIYNLLEESQNQQEKNEQELLELDKWANLWNWF

SEQ ID NO: 5 - protein, 36 aa, Human immunodeficiency virus type 1
YTSLIYSLLEKSQIQQEKNEQELLELDKWASLWNWF

SEQ ID NO: 6 - protein, 36 aa, Human immunodeficiency virus type 1
LEANISQSLEQAQIQQEKNMYELQKLNSWDVFTNWL

SEQ ID NO: 7 - protein, 41 aa, Human immunodeficiency virus type 1
CGGNNLLRAIEAQQHLLQLTVWGIKQLQARILAVERYLKDQ

SEQ ID NO: 8 - protein, 35 aa, Human immunodeficiency virus type 1
WMEWDREINNYTSLIGSLIEESQNQQEKNEQELLE

SEQ ID NO: 9 - protein, 48 aa, Human immunodeficiency virus type 1
YTSLIHSLIEESQNQQEKNEQELLELDKWASLWNWFNITNWLWLIKFI

SEQ ID NO: 10 - protein, 36 aa, Human immunodeficiency virus type 1
YTSLIHSLIEESQNQQEKNEQELLELDKWASLWNAF

SEQ ID NO: 11 - protein, 36 aa, Human immunodeficiency virus type 1
YTSLIHSLIEESQNQQEKNEQELLELDKWASLANWF

SEQ ID NO: 12 - protein, 36 aa, Human immunodeficiency virus type 1
YTSLIHSLIEESQNQQEKNEQQLLELDKWASLWNWF

SEQ ID NO: 13 - protein, 36 aa, Human immunodeficiency virus type 1
YTSLIHSLIEESQNQQEKNEQELLQLDKWASLWNWF

SEQ ID NO: 14 - protein, 36 aa, Human immunodeficiency virus type 1
YTSLIHSLIEESQNQQEKNQQELLQLDKWASLWNWF

SEQ ID NO: 15 - protein, 36 aa, Human immunodeficiency virus type 1
YTSLIHSLQEESQNQQEKNEQELLELDKWASLWNWF

SEQ ID NO: 16 - protein, 35 aa, Human immunodeficiency virus type 1
YTSLIHSLIEESQNQQEKNEQELLELDKWASLWNW

SEQ ID NO: 17 - protein, 36 aa, Human immunodeficiency virus type 1
YTSLIHSLIEQSQNQQEKNEQELLELDKWASLWNWF

SEQ ID NO: 18 - protein, 36 aa, Human immunodeficiency virus type 1
YTSLIHSLIQESQNQQEKNEQELLELDKWASLWNWF

SEQ ID NO: 19 - protein, 36 aa, Human immunodeficiency virus type 1; identical to SEQ ID NO: 12

SEQ ID NO: 20 - protein, 36 aa, Human immunodeficiency virus type 1
YTSLIQSLIEESQNQQEKNEQQLLELDKWASLWNWF

SEQ ID NO: 21 - protein, 36 aa, Human immunodeficiency virus type 1
YTSLIHSLIEESQNQQEKNEQELLELDKWASLFNFF

SEQ ID NO: 22 - protein, 36 aa, Human immunodeficiency virus type 1
YTSLIHSLIEESQNLQEKNEQELLELDKWASLWNWF

SEQ ID NO: 23 - protein, 36 aa, Human immunodeficiency virus type 1
YTSLIHSLIEESQNQQEKLEQELLELDKWASLWNWF

SEQ ID NO: 24 - protein, 36 aa, Human immunodeficiency virus type 1
YTSLIHSLIEESQNQQEKNEQELLEFDKWASLWNWF

SEQ ID NO: 25 - protein, 36 aa, Human immunodeficiency virus type 1
YTSLIHSLIEESQNQQEKNEQELLELDKWASPWNWF

SEQ ID NO: 26 - protein, 36 aa, Human immunodeficiency virus type 1
YTSLIHSLIEESQNQQEKNEQELLELDKWASLWNSF

SEQ ID NO: 27 - protein, 57 aa, Human immunodeficiency virus type 1
NKSLEQIWNNMTWMEWDREINNYTSLIHSLIEESQNQQEKNEQELLELDKASLWNWF

SEQ ID NO: 28 - protein, 36 aa, Human immunodeficiency virus type 1
SLEQIWNNMTWMEWDREINNYTSLIHSLIEESQNQQ

SEQ ID NO: 29 - protein, 36 aa, Human immunodeficiency virus type 1
LEQIWNNMTWMEWDREINNYTSLIHSLIEESQNQQE

SEQ ID NO: 30 - protein, 36 aa, Human immunodeficiency virus type 1
EQIWNNMTWMEWDREINNYTSLIHSLIEESQNQQEK

SEQ ID NO: 31 - protein, 36 aa, Human immunodeficiency virus type 1
QIWNNMTWMEWDREINNYTSLIHSLIEESQNQQEKN

SEQ ID NO: 32 - protein, 36 aa, Human immunodeficiency virus type 1
IWNNMTWMEWDREINNYTSLIHSLIEESQNQQEKNE

SEQ ID NO: 33 - protein, 36 aa, Human immunodeficiency virus type 1
WNNMTWMEWDREINNYTSLIHSLIEESQNQQEKNEQ

SEQ ID NO: 34 - protein, 36 aa, Human immunodeficiency virus type 1
NNMTWMEWDREINNYTSLIHSLIEESQNQQEKNEQE

SEQ ID NO: 35 - protein, 36 aa, Human immunodeficiency virus type 1
NMTWMEWDREINNYTSLIHSLIEESQNQQEKNEQEL

SEQ ID NO: 36 - protein, 47 aa, Human immunodeficiency virus type 1
MTWMEWDREINNYTSLIHSLIEESQNQQEKNEQELLELDKWASLWNW

SEQ ID NO: 37 - protein, 36 aa, Human immunodeficiency virus type 1
TWMEWDREINNYTSLIHSLIEESQNQQEKNEQELLE

SEQ ID NO: 38 - protein, 36 aa, Human immunodeficiency virus type 1
WMEWDREINNYTSLIHSLIEESQNQQEKNEQELLEL

SEQ ID NO: 39 - protein, 35 aa, Human immunodeficiency virus type 1
WMEWDREINNYTSLIHSLIEESQNQQEKNEQELLE

SEQ ID NO: 40 - protein, 36 aa, Human immunodeficiency virus type 1
MEWDREINNYTSLIHSLIEESQNQQEKNEQELLELD

SEQ ID NO: 41 - protein, 36 aa, Human immunodeficiency virus type 1
EWDREINNYTSLIHSLIEESQNQQEKNEQELLELDK

SEQ ID NO: 42 - protein, 36 aa, Human immunodeficiency virus type 1
WDREINNYTSLIHSLIEESQNQQEKNEQELLELDKW

SEQ ID NO: 43 - protein, 36 aa, Human immunodeficiency virus type 1
DREINNYTSLIHSLIEESQNQQEKNEQELLELDKWA

SEQ ID NO: 44 - protein, 36 aa, Human immunodeficiency virus type 1
REINNYTSLIHSLIEESQNQQEKNEQELLELDKWAS

SEQ ID NO: 45 - protein, 36 aa, Human immunodeficiency virus type 1
EINNYTSLIHSLIEESQNQQEKNEQELLELDKWASL

SEQ ID NO: 46 - protein, 36 aa, Human immunodeficiency virus type 1
INNYTSLIHSLIEESQNQQEKNEQELLELDKWASLW

SEQ ID NO: 47 - protein, 36 aa, Human immunodeficiency virus type 1
NYTSLIHSLIEESQNQQEKNEQELLELDKWASLWNW

SEQ ID NO: 48 - protein, 36 aa, Human immunodeficiency virus type 1
TSLIHSLIEESQNQQEKNEQELLELDKWASLWNWFN

SEQ ID NO: 49 - protein, 36 aa, Human immunodeficiency virus type 1
SLIHSLIEESQNQQEKNEQELLELDKWASLWNWFNI

SEQ ID NO: 50 - protein, 36 aa, Human immunodeficiency virus type 1
LIHSLIEESQNQQEKNEQELLELDKWASLWNWFNIT

SEQ ID NO: 51 - protein, 35 aa, Human immunodeficiency virus type 1
TSLIHSLIEESQNQQEKNEQELLELDKWASLWNWF

SEQ ID NO: 52 - protein, 33 aa, Human immunodeficiency virus type 1
LIHSLIEESQNQQEKNEQELLELDKWASLWNWF

SEQ ID NO: 53 - protein, 26 aa, Human immunodeficiency virus type 1
ESQNQQEKNEQELLELDKWASLWNWF

SEQ ID NO: 54 - protein, 23 aa, Human immunodeficiency virus type 1
NQQEKNEQELLELDKWASLWNWF

SEQ ID NO: 55 - protein, 20 aa, Human immunodeficiency virus type 1
EKNEQELLELDKWASLWNWF

SEQ ID NO: 56 - protein, 28 aa, Human immunodeficiency virus type 1
LRAIEAQQHLLQLTVWQIKQLQARILAV

SEQ ID NO: 57 - protein, 36 aa, Human immunodeficiency virus type 1; identical to SEQ ID NO: 5

SEQ ID NO: 58 - protein, 38 aa, Human immunodeficiency virus type 1
NNLLRAIEAQQGLLQLTVWGIKQLQARILAVERYLKDQ

SEQ ID NO: 59 - protein, 41 aa, Human immunodeficiency virus type 1
NNLLRAIEAQQHLLQLTVWGIKQLQARILAVERYLKDQGGC

SEQ ID NO: 60 - protein, 41 aa, Human immunodeficiency virus type 1; identical to SEQ ID NO: 59

SEQ ID NO: 61 - protein, 43 aa, Human immunodeficiency virus type 1
CGGNNLLRAIEAQQHLLQLTVWGIKQLQARILAVERYKDQGGC

SEQ ID NO: 62 - protein, 39 aa, Human immunodeficiency virus type 1
LSGIVQQQNNLLRAIEAQQHLLQLTVWGIKQLQARILAV

(19) United States
(12) Patent Application Publication
(10) Pub. No.: US 2005/0065319 A1
(43) Pub. Date: Mar. 24, 2005

(54) Combination method for treating viral infections

(76) Inventor: Bahige M. Baroudy, Westfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 10/168,295

(22) PCT Filed: Jun. 11, 2001
(86) PCT No.: PCT/US01/18802

Related U.S. Application Data
(60) Provisional application No. 60/256,657, filed on Dec. 19, 2000.

(51) Int. Cl. C07K 16/00; C07K 7/00; C07K 1/00; C07K 14/00; C07K 5/00; C07K 17/00; A01N 37/18; A61K 38/00

(52) U.S. Cl. 530/324; 530/350; 514/2

(57) ABSTRACT

This invention provides novel combination therapies comprising a CCR5 antagonist, or a pharmaceutically acceptable salt thereof, and a DP-178 polypeptide, or a pharmaceutically acceptable derivative thereof, for the treatment of HIV. The present combination permits a more tolerable treatment schedule by reducing the frequency of administration of a DP-178 polypeptide from twice daily subcutaneously to once daily or even just a few times per week.

### FIELD OF THE INVENTION

This invention relates to combination therapies and to methods for treating viral infections, e.g., human immunodeficiency virus (HIV).

### BACKGROUND OF THE INVENTION

The global health crisis caused by HIV, the causative agent of Acquired Immunodeficiency Syndrome (AIDS), is unquestioned, and while recent advances in drug therapies have been successful in slowing the progression of AIDS, there is still a need to find a safer, more efficient, less expensive way to control the virus.

HIV infection begins by attachment of the virus to a target cell membrane through interaction with the cellular receptor CD4 and a secondary chemokine co-receptor molecule. It proceeds by replication and dissemination of infected cells through the blood and other tissue. There are various chemokine receptors, but for macrophage-tropic HIV, believed to be the key pathogenic strain that replicates in vivo in the early stages of infection, the principal chemokine receptor required for the entry of HIV into the cell is CCR5. It has also been reported that the CCR5 gene plays a role in resistance to HIV infection, since individuals containing a homozygous deletion for the CCR5 gene are largely resistant to HIV infection. Therefore, interfering with the interaction between the viral receptor CCR5 and HIV may block viral entry into the cell without any significant side effects.

Attention has also been given to HIV-1 envelope proteins, e.g., gp160, gp120 and gp41, which are major antigens for anti-HIV antibodies present in AIDS patients. U.S. Pat. Nos. 5,464,933 and 6,020,459 and International Patent Publication WO 96/40191 describe a portion of the HIV gp41 protein (known as DP-178, T-20, and pentafuside), which is required for the virus to gain entry into CD4⁺ cells. This protein fragment may prevent HIV from gaining entry into host CD4⁺ cells and further prevent cell to cell transmission of the virus.

T-20 has been administered as a continuous infusion or twice daily (bid) subcutaneously, typically in dosages of 100 mg/day. This greatly inconveniences the lifestyle of a patient receiving this peptide therapy, since the patient undergoing this anti-HIV therapy must visit a clinician twice a day to receive injections of the peptide.

Vandamme et al. [Antiviral Chemistry & Chemotherapy 9:187-203 (1998)] disclose current clinical treatments of HIV-1 infections in man including at least triple drug combinations or so-called Highly Active Antiretroviral Therapy (“HAART”); HAART involves various combinations of nucleoside reverse transcriptase inhibitors, non-nucleoside reverse transcriptase inhibitors and HIV protease inhibitors. In compliant drug-naive patients, HAART is effective in reducing mortality and progression of HIV-1 to AIDS. However, these multidrug therapies do not eliminate HIV-1 and long-term treatment usually results in multidrug resistance. Furthermore, HIV therapies are expensive, costing tens of thousands of dollars per year. It would be an advantage to provide an anti-HIV therapy which blocks fusion and entry of HIV into CD4⁺ cells by different mechanisms, has a more convenient administration schedule, and as good if not a greater efficacy against HIV compared with known treatments, having lower costs. Thus, development of new drug therapies to provide better HIV-1 treatment remains a priority.

### SUMMARY OF THE INVENTION

This invention provides an improved treatment for viral infections, and, in particular, HIV infection. It provides novel drug combination therapies, comprising an effective amount of a first antiviral agent, which is a piperidine derivative of structural formula I or II, or pharmaceutically acceptable salts thereof, and an effective amount of a second antivrial agent which is a DP-178 polypeptide, or a pharmaceutically acceptable derivative thereof. A combination of the present invention is administered to an individual infected with a virus, preferably HIV, using a dosage and administration schedule adequate to inhibit viral activity, viral expression, viral transmission, or reduce viral load. A combination therapy of the present invention preferably provides a therapy having improved efficacy over other known anti-HIV combination therapies or therapies which contain only one of the antiviral agents in the present combination. More preferably, a combination therapy of the present invention is a synergistic combination.

It is an advantage of this invention that a present combination therapy allows the clinician to decrease the frequency of administration of a DP-178 polypeptide (i.e., less than bid), and, if desired, to administer the combination with one or more additional therapeutic compounds as described, infra.

Thus, in one embodiment, the invention provides a method of treating an HIV infection comprising administering in combination a therapeutically effective amount of a CCR5 antagonist, or a pharmaceutically acceptable salt thereof, and a therapeutically effective amount of a DP-178 polypeptide, or a pharmaceutically acceptable derivative thereof.

In another embodiment, the present invention provides a method of treating an HIV infection comprising administering a CCR5 antagonist of structural formula 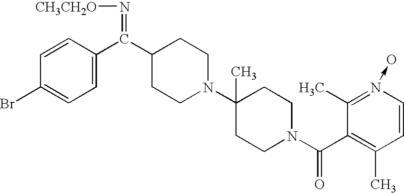
or a pharmaceutically acceptable salt thereof,
and T-20,
   wherein the CCR5 antagonist is administered one to two times a day in dosages of from 25 to 400 mg/day, and the T-20 is administered once (qd) every other day or once (qd) two, three or four times per week in a dosage of from 3 to 200 mg, or a multiple thereof to reduce the viral load in the infected individual by 1 or 2 logs.

In another aspect, the present invention provides a kit containing single package pharmaceutical compositions for use in combination to treat HIV infection, which comprises in a first container a pharmaceutical composition comprising a CCR5 antagonist, or pharmaceutically acceptable salt thereof, in a pharmaceutically acceptable carrier, in an oral dosage from 25 to 600 mg to be administered from 1 to 3 times per week or every other day, and in a second container a pharmaceutical composition comprising a DP-178 polypeptide, or a pharmaceutically acceptable derivative thereof, in a pharmaceutically acceptable carrier, in a subcutaneous dosage from 3 to 200 mg, or a multiple thereof which reduces the viral load by 1 or 2 logs.

### DETAILED DESCRIPTION OF THE INVENTION

The term “viral infection” is used to describe a diseased state, which can be latent, where a virus invades a cell, uses the cell's reproductive machinery to multiply or replicate, and ultimately lyses the cell causing cell death and release of progeny virus particles followed by further infection of other cells by the progeny.

The terms “treating” or “preventing” used in relation to a viral infection means to inhibit viral activity, expression, replication or transmission of a virus, or to prevent the virus from establishing itself in a host cell, and which results in an amelioration or alleviation of the symptoms of the disease caused by the viral infection. A treatment or therapy is considered therapeutic if there is a reduction in viral load or decrease in mortality or morbidity.

A “therapeutically effective amount” of a DP-178 polypeptide or a CCR5 antagonist compound, or their derivatives, is an amount sufficient to treat or prevent a viral infection and according to a suitable administration schedule, i.e., the amount and dosaging schedule exhibits antiviral activity, thereby lowering HIV RNA plasma levels in the serum of an infected individual to less than 500 copies per ml of serum, preferably to less than 200 copies per ml of serum, more preferably to less than 50 copies per ml of serum, and most prefereably the number of copies is undetectable, as measured by quantitative, multi-cycle reverse transcriptase PCR methodology. HIV RNA is preferably measured using the methodology of Amplicor-1 Monitor 1.5 (available from Roche Diagnsotics) or of Nuclisens HIV-1 QT-1.

The term “combination therapy” refers to a therapy for treating viral infections, preferably HIV, which includes administration of an effective amount of a CCR5 antagonist and a DP-178 polypeptide. A combination therapy of this invention may include one or more antiviral agents, e.g., HAART. In addition, a combination therapy of this invention can be used as a prophylactic measure in previously uninfected individuals after a possible acute exposure to an HIV virus. Examples of such prophylactic use of the peptides may include, but are not limited to, prevention of virus transmission from mother to infant and other settings where the likelihood of HIV transmission exists, such as, for example, accidents in health care settings wherein workers are exposed to HIV-containing blood products.

The term “synergistic” refers to a combination which is more effective than the additive effects of any two or more single agents. A “synergistic effect” refers to the ability to use lower amounts or dosages of antiviral agents in a single therapy to treat or prevent viral infection. The lower doses typically result in a decreased toxicity without reduced efficacy. In addition, a synergistic effect can improve efficacy, e.g., improved antiviral activity, or avoid or reduce the extent of any viral resistance against an antiviral agent. A synergistic effect between a DP-178 polypeptide, or a pharmaceutically acceptible derivative thereof, and a CCR5 antagonist compound, or a pharmaceutically acceptable salt thereof, can be determined from conventional antiviral assays, e.g., as described infra. The results of an assay can be analyzed using Chou and Talalay's combination method to obtain a Combination Index (Chou and Talalay, 1984, Adv. Enzyme Regul. 22:27-55) and ‘Dose Effect Analysis with Microcomputers’ software (Chou and Chou, 1987, Software and Manual. p19-64. Elsevier Biosoft, Cambridge, UK). A Combination Index value of less than 1 indicates synergy, greater than 1 indicates antagonism and equal to 1 indicates an additive effect. The results of these assays can also be analyzed using the method of Pritchard and Shipman (Pritchard and Shipman, 1990, Antiviral Research 14: 181-206).

The term “pharmaceutically acceptable carrier” refers to a carrier medium that does not interfere with the effectiveness of the biological activity of the active ingredient, is chemically inert and is generally not toxic to the recipient.

The term “pharmaceutically acceptable derivative” refers to a tuncation, analog or other modification of a polypeptide, which exhibits antiviral activity and is generally non-toxic.

The term “antiviral activity” refers to an inhibition of HIV transmission to uninfected CD4⁺ cells, inhibition of the replication of HIV, prevention of HIV from establishing itself in a host, or ameliorating or alleviating the symptoms of the disease caused by HIV infection. These effects can be evidenced by a reduction in viral load or decrease in mortality and/or morbidity, which assays are described infra. An antiviral agent, or anti-HIV-1 drug, has antiviral activity and is useful for treating HIV-1 infections alone, or as part of a multi-drug combination therapy, e.g., the HAART triple and quadruple combination therapies.

A “therapeutic agent” is any molecule, compound or therapy that improves the treatment of a viral infection or the diseases caused thereby. Preferably, the therapeutic agent has antiviral activity.

Viruses whose transmission may be inhibited by the antiviral activity of a combination therapy of this invention include, for example: human retroviruses, particularly HIV-1 and HIV-2 and the human T-lymphocyte viruses (HTLV-I and II); non-human retroviruses, including bovine leukosis virus, feline sarcoma and leukemia viruses, simian immunodeficiency, sarcoma and leukemia viruses, and sheep progress pneumonia viruses; non-retroviral viruses, including human respiratory syncytial virus, canine distemper virus, newcastle disease virus, human parainfluenza virus, influenza viruses, measles viruses, Epstein-Barr viruses, hepatitis B viruses, and simian Mason-Pfizer viruses; and non-enveloped viruses, including picornaviruses such as polio viruses, hepatitis A virus, enterovirus, echoviruses and coxsackie viruses, papovaviruses such as papilloma virus, parvoviruses, adenoviruses and reoviruses.

Piperidine Derivative CCR5 Antagonist Compounds

Compounds having the structural formulas I and II below, and pharmaceutically acceptable salts thereof, are collectively referred to herein as “CCR5 antagonists”. These compounds antagonize the CC chemokine receptor 5, and are described in U.S. patent application Ser. Nos. 09/562,815 and 09/562,814 and in WO 00/66559 and WO 00/11632, which are each incorporated herein by reference in their entireties.

In one embodiment, a combination therapy of this invention comprises a compound of structural formula I, or a pharmaceutically acceptable salt thereof:

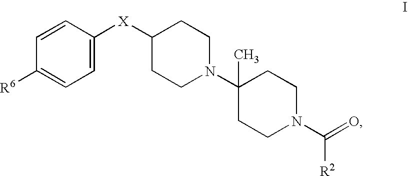

wherein R⁶, X and R² are as defined in Table 1:

TABLE 2

| R⁶ | X | R² |
|---|---|---|
| Br | 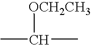 | 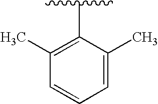 |
| Br | 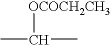 | 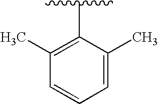 |
| Br | 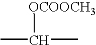 | 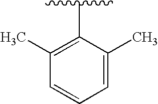 |
| Br | 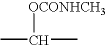 | 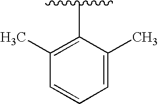 |
| Br | 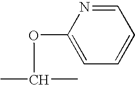 | 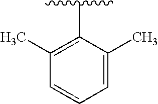 |
| Br | 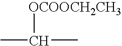 | 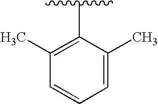 |
| Br | 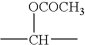 | 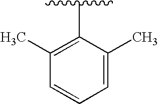 |
| Br | 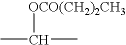 | 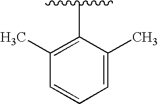 |
| Br | 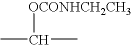 | 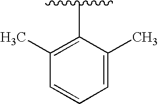 |
| Br | 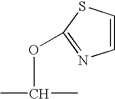 | 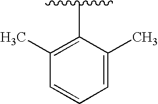 |
| Br | 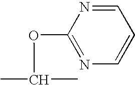 | 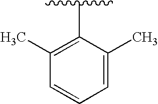 |
| Br | 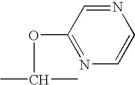 | 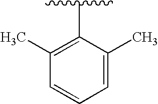 |
| CH₃SO₂— | 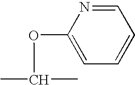 | 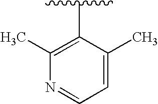 |
| Br | 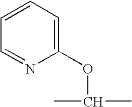 | 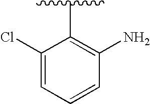 |
| Br | 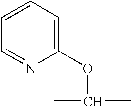 | 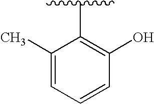 |
| Br | 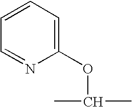 | 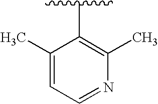 |
| Br | 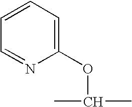 | 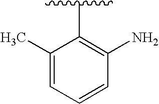 |
| H₃CSO₂— | 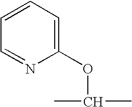 | 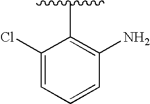 |
| H₃CSO₂— | 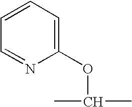 | 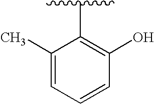 |
| F₃C— | 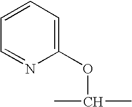 | 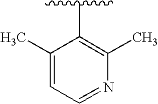 |
| H₃CSO₂— | 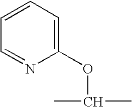 | 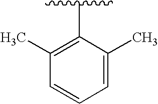 |
| H₃CSO₂— | 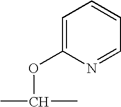 | 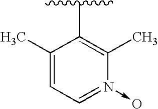 |
| F₃CO— | 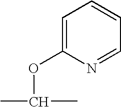 | 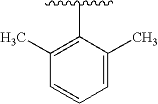 |
| F₃CO— | 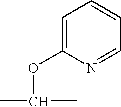 | 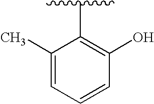 |
| F₃CO— | 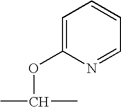 | 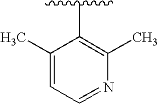 |
| Br | 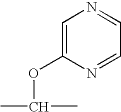 | 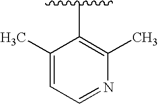 |
| Br | 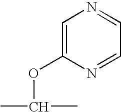 | 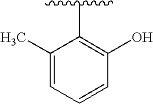 |
| F₃CO— | 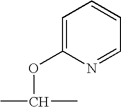 | 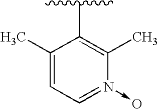 |
| Br | 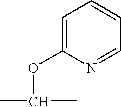 | 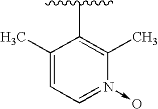 |
| Br | 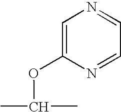 | 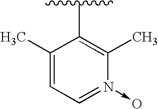 |
| H₃CSO₂— | 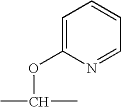 | 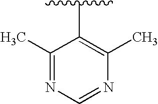 |
| H₃CSO₂— | 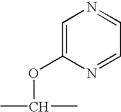 | 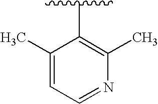 |
| H₃CSO₂— | 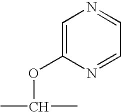 | 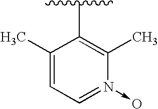 |
| H₃CSO₂— | 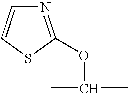 | 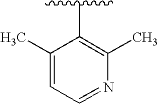 |
| H₃CSO₂— | 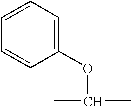 | 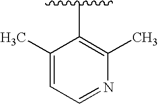 |
| H₃CSO₂— | 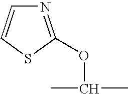 | 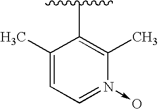 |
| F₃C— | 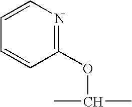 | 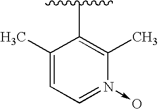 |
| F₃CO— | 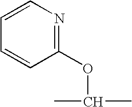 | 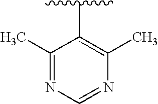 |
| F₃CO— | 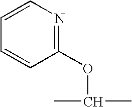 | 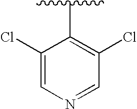 |
| Cl | 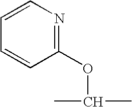 | 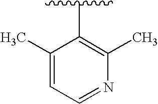 |
| Cl | 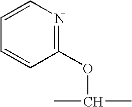 | 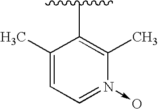 |
| Cl | 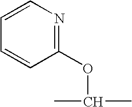 | 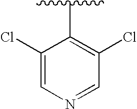 |
| Cl | 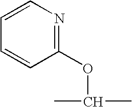 | 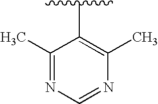 |
| Br | 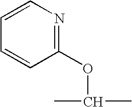 | 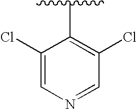 |
| H₃CSO₂— | 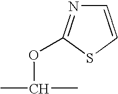 | 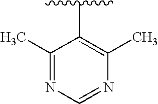 |
| F₃C— | 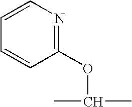 | 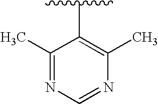 |
| H₃CSO₂— | 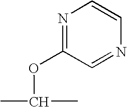 | 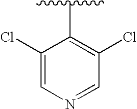 |
| H₃CSO₂— | 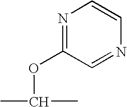 | 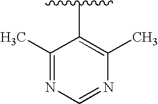 |
| F₃C— | 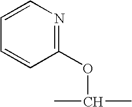 | 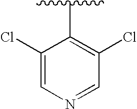 |
| F | 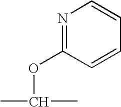 | 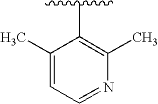 |
| F | 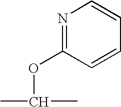 | 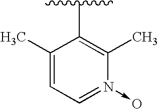 |
| F | 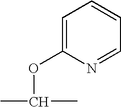 | 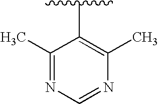 |
| Cl | 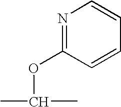 | 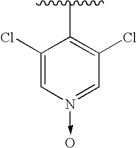 |
| F | 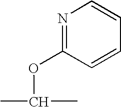 | 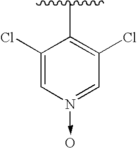 |
| Br | 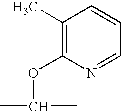 | 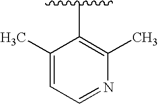 |
| Br | 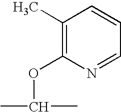 | 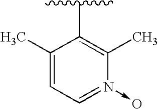 |
| Br | 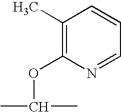 | 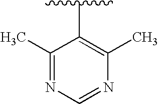 |
| Br | 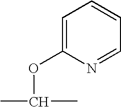 | 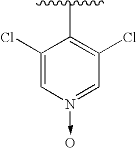 |
| F₃C— | 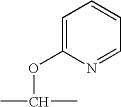 | 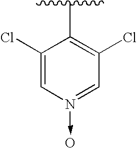 |
| Br | 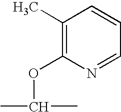 | 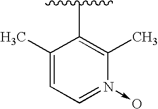 |
| Br | 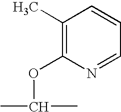 | 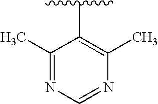 |
| Br | 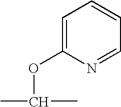 | 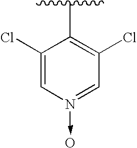 |
| F₃C— | 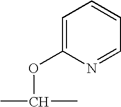 | 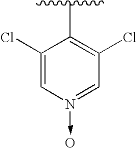 |
| F₃C— | 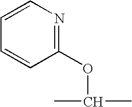 | 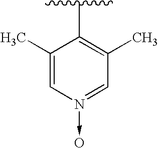 |
| F₃C— | 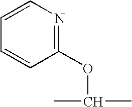 | 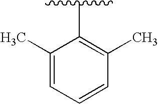 |
| F | 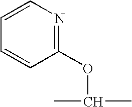 | 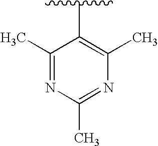 |
| Br | 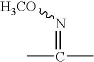 | 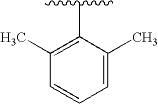 |
| Br | 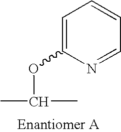 | 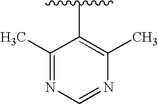 |
| Br | 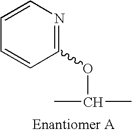 | 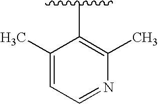 |
| Br | 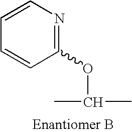 | 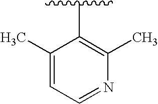 |
| Br | 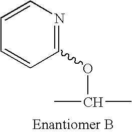 | 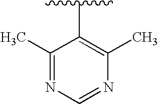 |
| F₃CO— | 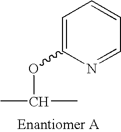 | 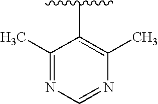 |
| F₃CO— | 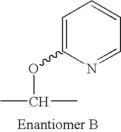 | 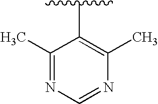 |
| F₃CO— | 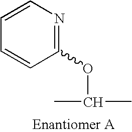 | 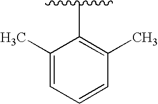 |
| F₃CO— | 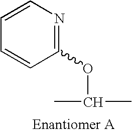 | 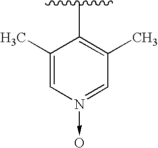 |
| F₃CO— | 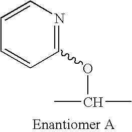 | 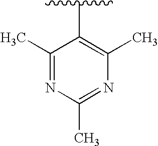 |
| F₃CO— | 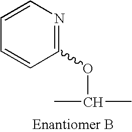 | 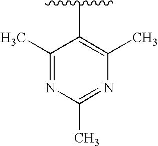 |
| Cl | 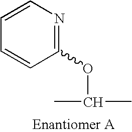 | 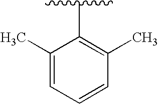 |
| Cl | 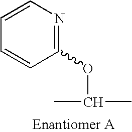 | 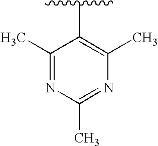 |
| Cl | 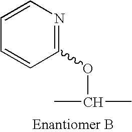 | 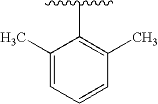 |
| Cl | 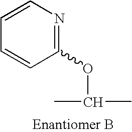 | 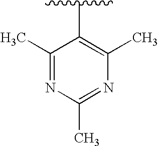 |
| F₃CO— | 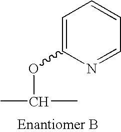 | 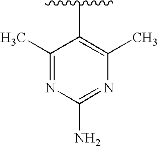 |
| Br | 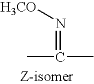 | 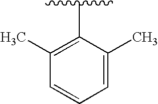 |
| Br | 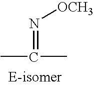 | 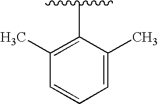 |
| Br | 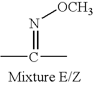 | 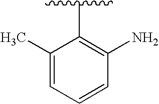 |
| Br | 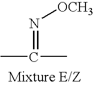 | 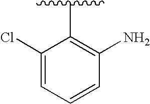 |
| Br | 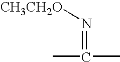 | 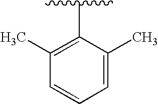 |
| Br | 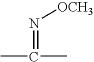 | 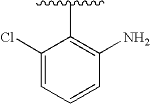 |
| Br | 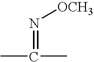 | 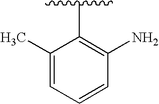 |
| Br | 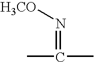 | 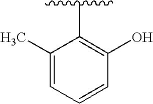 |
| Bt | 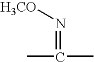 | 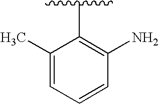 |
| Br | 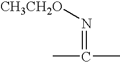 | 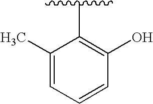 |
| Br | 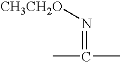 | 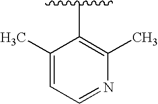 |
| Br | 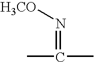 | 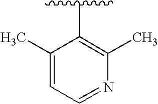 |
| Br | 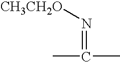 | 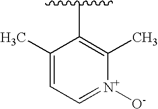 |
| Br | 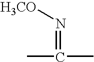 | 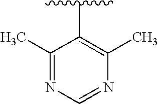 |
| Br | 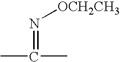 | 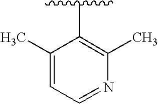 |
| Br | 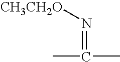 | 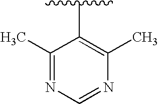 |
| Br | 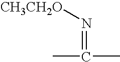 | 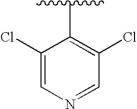 |
| Br | 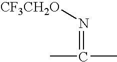 | 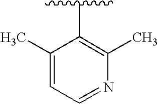 |
| Br | 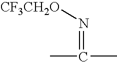 | 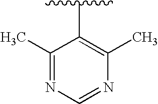 |
| Br | 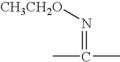 | 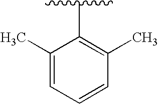 |
| Br | 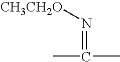 | 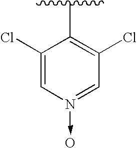 |
| Br | 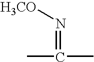 | 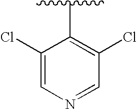 |
| Br | 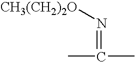 | 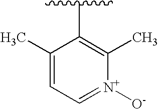 |
| Br | 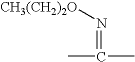 | 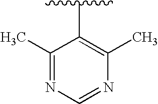 |
| Br | 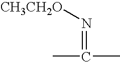 | 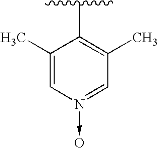 |
| Br | 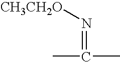 | 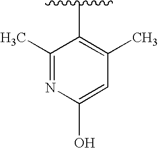 |
| Br | 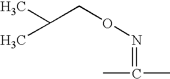 | 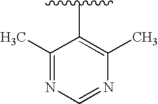 |
| Br | 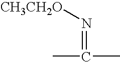 | 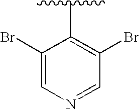 |
| Br | 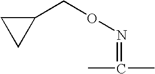 | 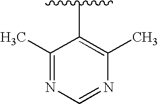 |
| Br | 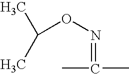 | 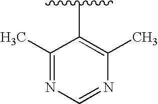 |
| Br | 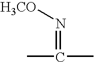 | 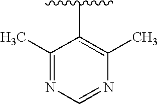 |
| Br | 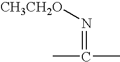 | 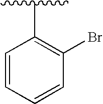 |
| Br | 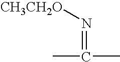 | 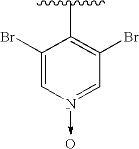 |
| Br | 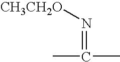 | 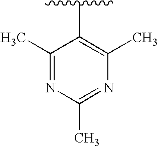 |
| F₃C— | 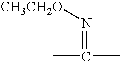 | 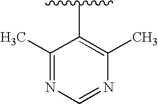 |
| Br | 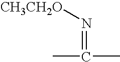 | 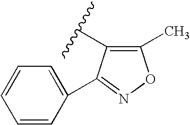 |
| Br | 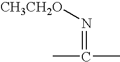 | 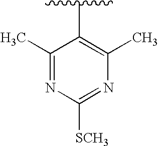 |
| Br | 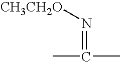 | 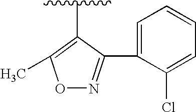 |
| Br | 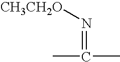 | 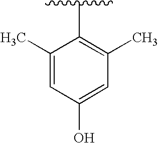 |
| Br | 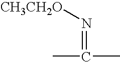 | 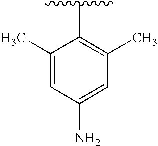 |
| Br | 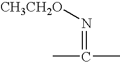 | 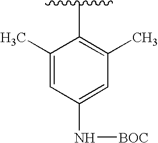 |
| Br | 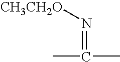 | 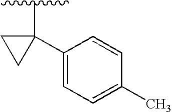 |
| Br | 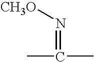 | 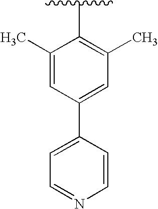 |
| Br | 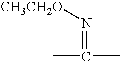 | 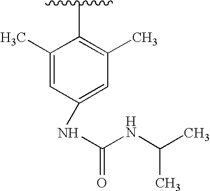 |
| F₃CO— | 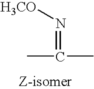 | 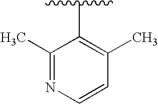 |
| F₃CO— | 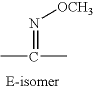 | 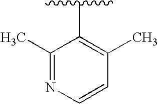 |
| Br | 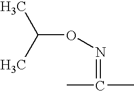 | 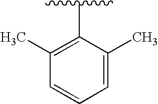 |
| Br | 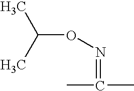 | 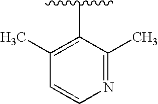 |
| Br | 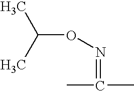 | 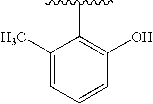 |
| Br | 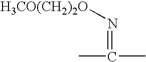 | 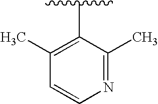 |
| Br | 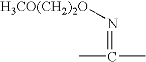 | 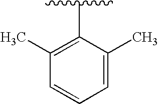 |
| Br | 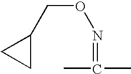 | 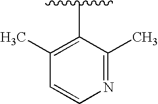 |
| F₃CO— | 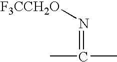 | 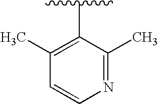 |
| F₃CO— | 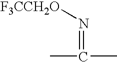 | 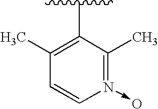 |
| F₃CO— | 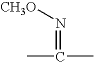 | 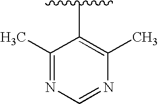 |
| F₃CO— | 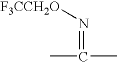 | 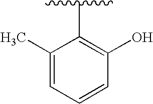 |
| F₃CO— | 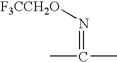 | 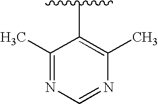 |
| F₃CO— | 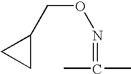 | 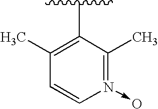 |
| Cl | 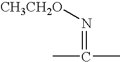 | 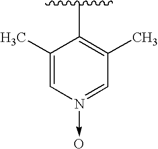 |
| Cl | 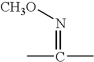 | 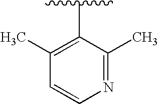 |
| F₃C— | 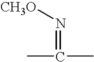 | 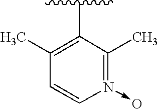 |
| Cl | 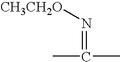 | 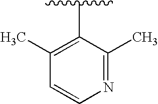 |
| Cl | 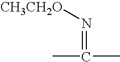 | 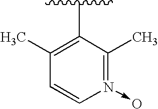 |
| Cl | 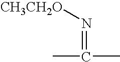 | 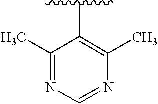 |
| F₃C— | 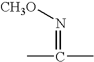 | 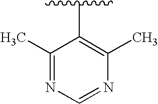 |
| Cl | 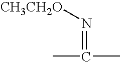 | 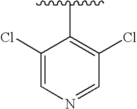 |
| F₃C— | 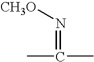 | 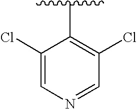 |
| F₃C— | 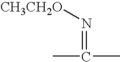 | 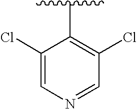 |
| Cl | 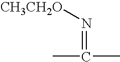 | 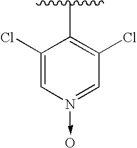 |
| Cl | 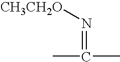 | 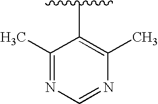 |
| F₃CO— | 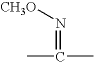 | 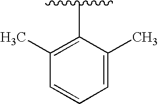 |
| F₃CO— | 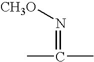 | 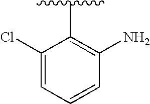 |
| F₃CO— | 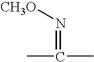 | 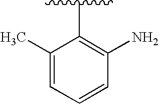 |
| F₃CO— | 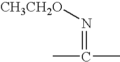 | 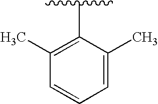 |
| F₃C— | 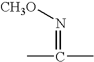 | 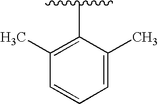 |
| F₃CO— | 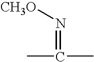 | 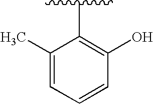 |
| F₃C— | 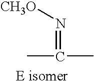 | 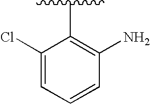 |
| F₃C— | 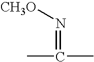 | 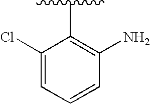 |
| F₃C— | 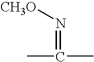 | 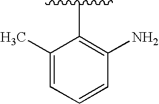 |
| F₃CO— | 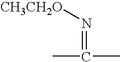 | 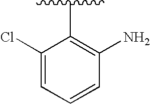 |
| F₃C— | 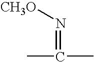 | 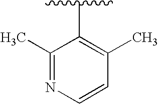 |
| F₃C— | 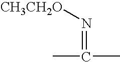 | 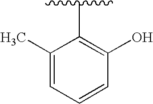 |
| F₃CO— | 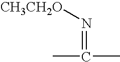 | 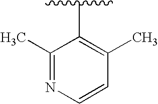 |
| F₃CO— | 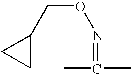 | 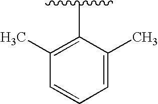 |
| F₃CO— | 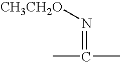 | 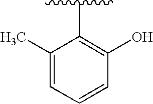 |
| F₃C— | 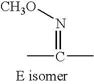 | 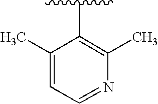 |
| F₃CO— | 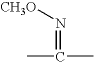 | 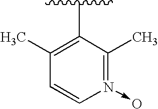 |
| F₃CO— | 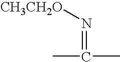 | 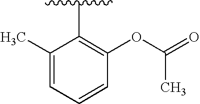 |
| F₃C— | 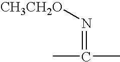 | 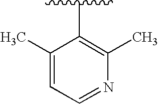 |
| F₃C— | 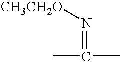 | 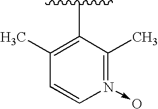 |
| F₃CO— | 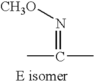 | 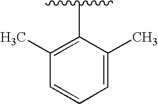 |
| F₃CO— | 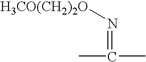 | 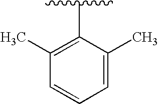 |
| F₃CO— | 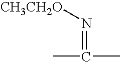 | 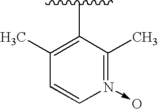 |
| F₃CO— | 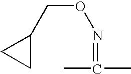 | 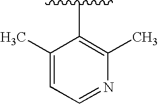 |
| F₃CO— | 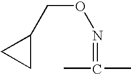 | 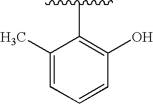 |
| F₃CO— | 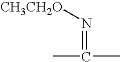 | 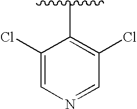 |
| F₃CO— | 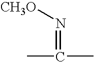 | 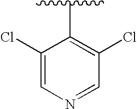 |
| F₃CO— | 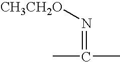 | 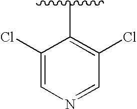 |
| F₃CO— | 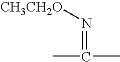 | 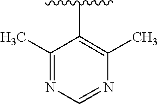 |
| F₃CO— | 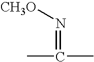 | 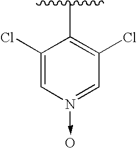 |
| F₃CO— | 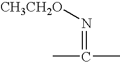 | 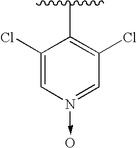 |
| F₃CO— | 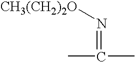 | 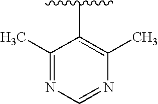 |
| F₃CO— | 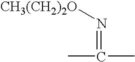 | 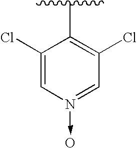 |
| F₃CO— | 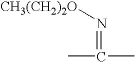 | 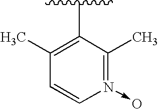 |
| Br | 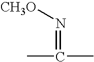 | 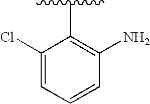 |
| F₃C— | 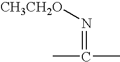 | 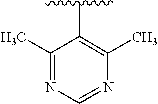 |
| Br | —CH₂— | 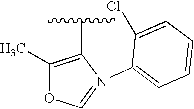 |
| Br | —CH₂— | 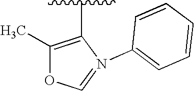 |
| Br | —CH₂— | 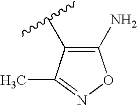 |
| Br | —CH₂— | 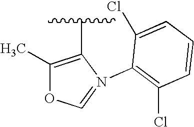 |
| Br | —CH₂— | 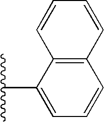 |
| Br | —CH₂— | 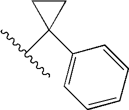 |
| Br | 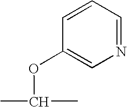 | 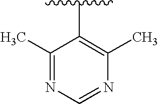 |
| CH₃SO₂— | 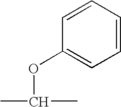 | 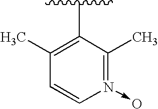 |
| Br | 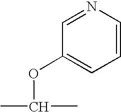 | 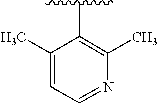 |
| Br | 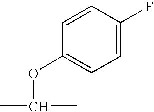 | 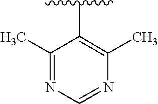 |
| F | 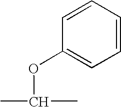 | 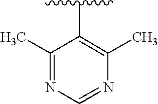 |
| F | 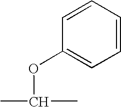 | 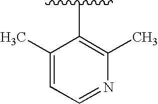 |
| F | 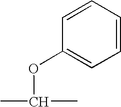 | 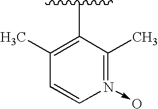 |
| Br | 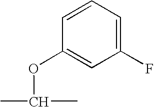 | 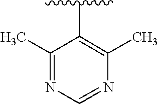 |
| Cl | 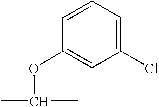 | 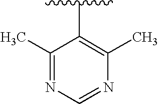 |
| F₃C— | 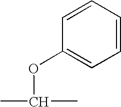 | 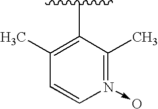 |
| CH₃SO₂— | 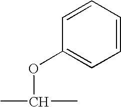 | 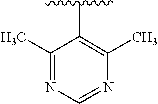 |
| CH₃SO₂— | 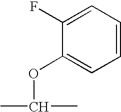 | 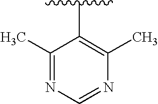 |
| F₃CO— | 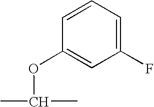 | 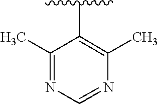 |
| F₃CO— | 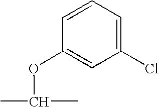 | 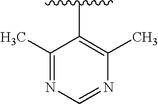 |
| CH₃SO₂— | 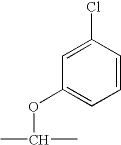 | 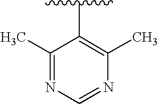 |
| CH₃SO₂— | 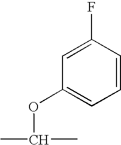 | 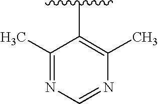 |
| F₃C— | 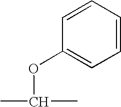 | 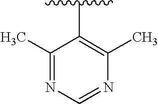 |
| F₃CO— | 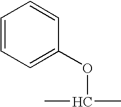 | 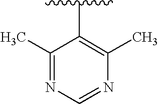 |
| F₃CO— | 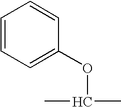 | 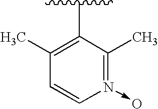 |
| F₃C— | 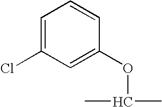 | 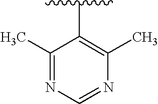 |
| H | 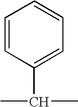 | 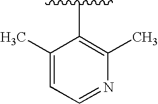 |
| F₃CO— | 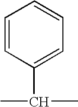 | 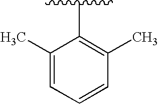 |
| F₃CO— | 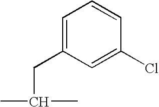 | 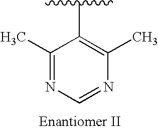 |
| F₃CO— | 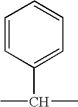 | 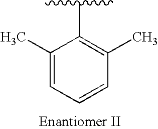 |
| F₃CO— | 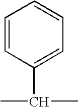 | 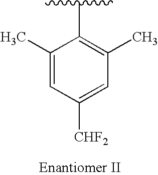 |
| F₃CO— | 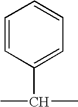 | 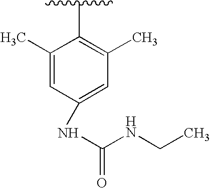 |
| CH₃SO₂— | 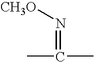 | 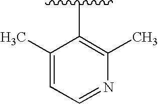 |
| CH₃SO₂— | 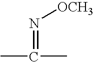 | 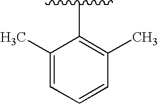 |
| CH₃SO₂— | 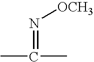 | 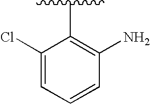 |
| CH₃SO₂— | 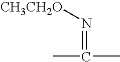 | 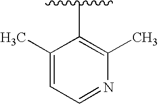 |
| CH₃SO₂— | 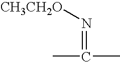 | 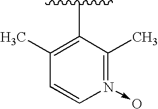 |
| CH₃SO₂— | 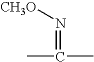 | 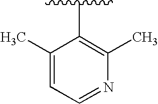 |
| CH₃SO₂— | 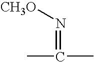 | 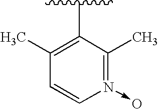 |
| CH₃SO₂— | 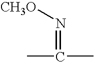 | 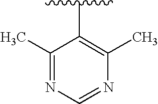 |

In a specific embodiment, a combination therapy of this invention comprises a compound of the structural formula 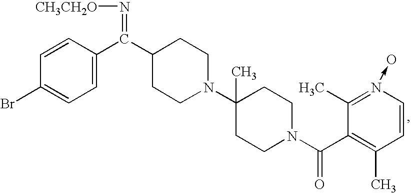
or a pharmaceutically acceptable salt thereof.

In another embodiment, a CCR5 antagonist compound used in a combination therapy of this invention has the structural formula II, or a pharmaceutically acceptable salt thereof:

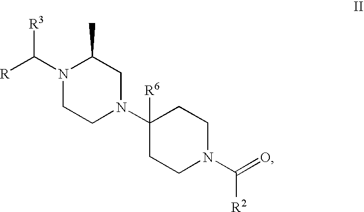

wherein R, R³, R⁶ and R² are as defined in Table 2:

TABLE 2

| R | R³ | R⁶ | R² |
|---|---|---|---|
| 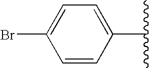 | 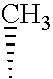 | H | 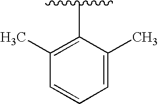 |
| 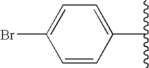 | 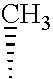 | —CH₃ | 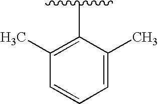 |
| 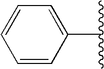 | 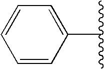 | H | 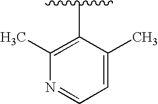 |
| 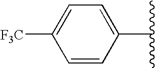 | 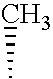 | H | 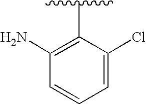 |
| 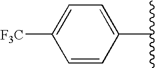 | 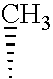 | H | 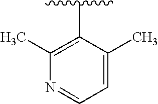 |
| 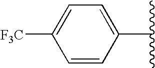 | 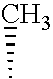 | H | 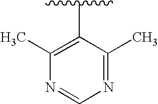 |
| 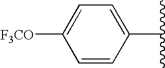 | 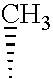 | —CH₃ | 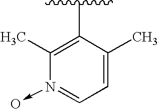 |
| 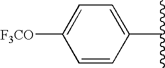 | 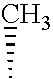 | —CH₃ | 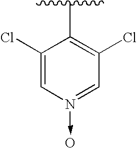 |
| 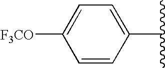 | 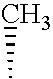 | —CH₃ | 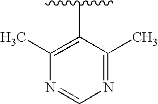 |
| 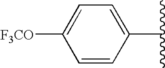 | 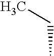 | —CH₃ | 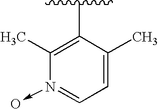 |
| 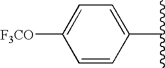 | 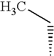 | —CH₃ | 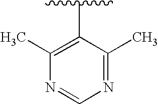 |
| 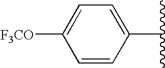 | 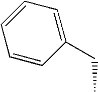 | —CH₃ | 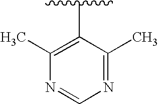 |
| 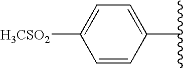 | 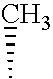 | H | 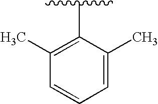 |
| 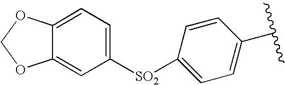 | 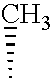 | H | 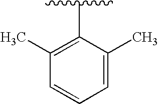 |
| 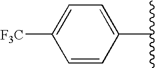 | 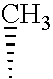 | —CH₃ | 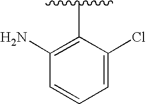 |
| 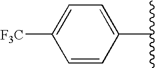 | 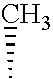 | —CH₃ | 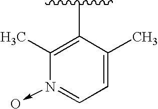 |
| 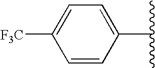 | 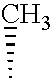 | —CH₃ | 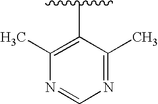 |
| 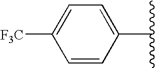 | 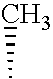 | —CH₃ | 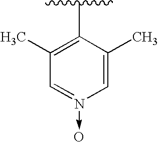 |
| 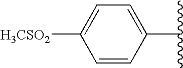 | 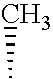 | —CH₃ | 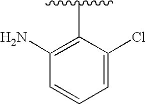 |
| 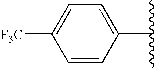 | H | —CH₃ | 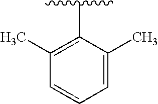 |
| 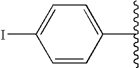 | H | —CH₃ | 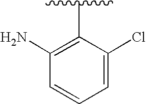 |
| 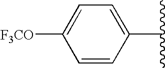 | H | —CH₃ | 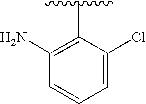 |
| 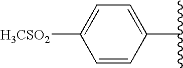 | H | —CH₃ | 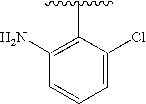 |
| 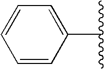 | 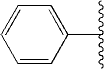 | —CH₃ | 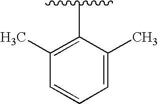 |
| 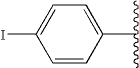 | H | H | 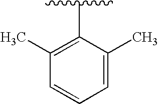 |
| 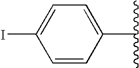 | 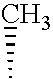 | H | 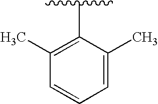 |
| 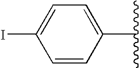 | 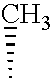 | —CH₃ | 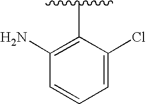 |
| 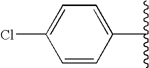 | 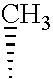 | H | 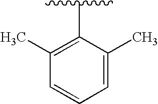 |
| 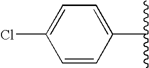 | 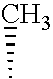 | —CH₃ | 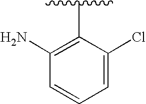 |
| 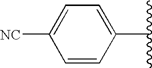 | 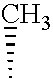 | H | 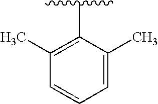 |
| 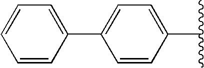 | 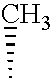 | H | 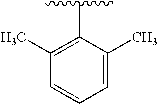 |
| 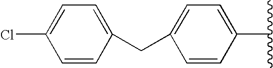 | 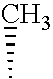 | H | 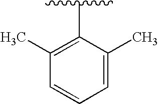 |
| 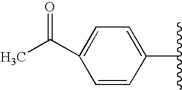 | 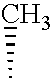 | H | 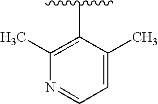 |
| 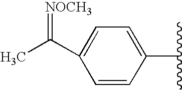 | 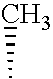 | —CH₃ | 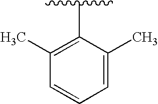 |
| 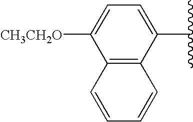 | H | —CH₃ | 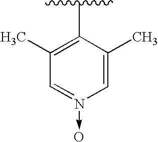 |
| 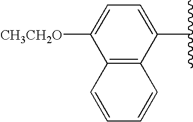 | H | —CH₃ | 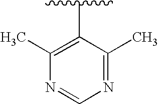 |
| 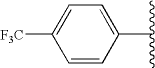 | 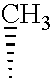 | —CH₂CH₃ | 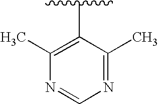 |
| 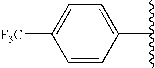 | 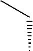 | —CH₂CH₃ | 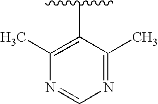 |
| 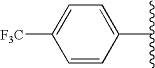 | 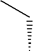 | —CH₂CH₃ | 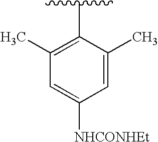 |
| 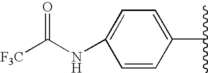 | 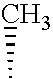 | —CH₃ | 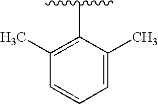 |
| 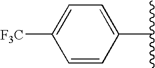 | 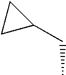 | —CH₃ | 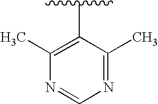 |
| 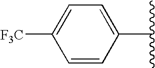 | 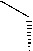 | —CH₃ | 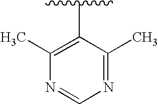 |
| 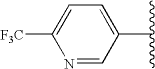 | 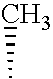 | —CH₃ | 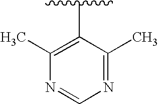 |
| 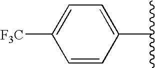 | 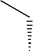 | —CH₃ | 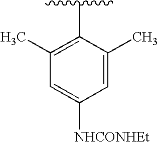 |
| 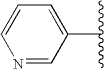 | 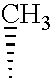 | —CH₃ | 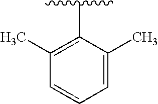 |
| 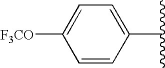 | 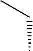 | —CH₃ | 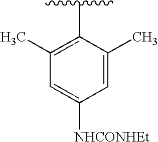 |
| 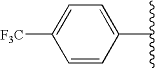 | 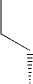 | —CH₃ | 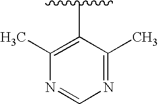 |
| 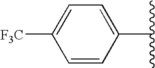 | 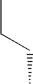 | —CH₃ | 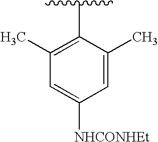 |
| 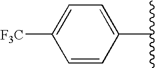 | 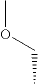 | —CH₃ | 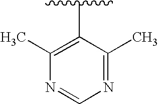 |
| 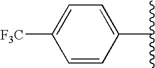 | 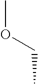 | —CH₃ | 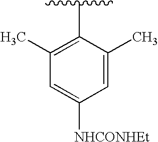 |
| 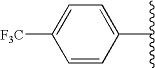 | 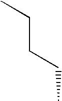 | —CH₃ | 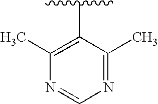 |
| 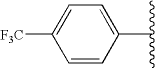 | 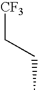 | —CH₃ | 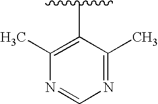 |

In a specific embodiment, a combination therapy of this invention comprises a compound of the structural formula: 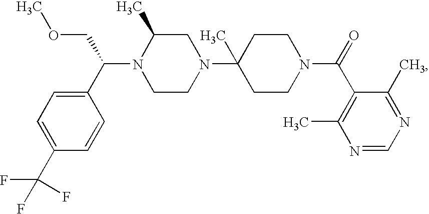
or pharmaceutically acceptable salt thereof.

Certain CCR5 antagonist compounds used in this invention may exist in different isomeric forms, e.g., enantiomers, diastereoisomers and atropisomers. The invention contemplates all such isomers both in pure form and in admixture, including racemic mixtures.

Certain compounds will be acidic in nature, e.g., compounds which possess a carboxyl or phenolic hydroxyl group. These compounds may form pharmaceutically acceptable salts. Examples of such salts include sodium, potassium, calcium, aluminum, gold and silver salts. Also contemplated are salts formed with pharmaceutically acceptable amines such as ammonia, alkyl amines, hydroxyalkylamines, N-methylglucamine, and the like.

Certain basic compounds also form pharmaceutically acceptable salts, e.g., acid addition salts. For example, the pyrido-nitrogen atoms may form salts with strong acid, while compounds having basic substituents such as amino groups also form salts with weaker acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous NaOH, potassium carbonate, ammonia and sodium bicarbonate. The free base forms differ from their respective salt forms somewhat in certain physical properties, such as solubility in polar solvents, but the acid and base salts are otherwise equivalent to their respective free base forms for purposes of the invention.

All such acid and base salts are intended to be pharmaceutically acceptable salts within the scope of the invention and all acid and base salts are considered equivalent to the free forms of the corresponding compounds for purposes of the invention.

A CCR5 antagonist compound used in this invention can be made according to procedures known in the art, e.g., using the methods described in U.S. Pat. No. 5,883,096, U.S. Pat. No. 6,037,352, U.S. Pat. No. 5,889,006, U.S. Pat. No. 5,952,349, and U.S. Pat. No. 5,977,138, which are incorporated herein by reference in their entireties. U.S. patent application Ser. No. 09/562,815 and 09/562,814 and WO 00/66559 and WO 00/11632 provide specific methodologies for making CCR5 antagonists used in this invention.

DP-178 Polypeptides

DP-178 polypeptides (i.e., full-length DP-178 and pharmaceutically acceptable derivatives thereof) are described in U.S. Pat. No. 5,464,933, U.S. Pat. No. 6,020,459 and WO 96/40191, which are each incorporated herein by reference in their entireties. The DP-178 region of viral gp41 may mediate fusion of HIV and the target cell membrane, by forming a complex with a distal region on gp41 (possibly DP-107) needed for the virus to fuse with the target cell. A DP-178 polypeptide used in this invention exhibits antifusogenic activity, (inhibiting virus fusion with a host cell membrane) antiviral activity, and/or the ability to modulate intracellular processes involving coil-coil peptide structures, and is identified or recognized by the ALLMOTI5, 107X178X4 and PLZIP search motifs described in U.S. Pat. No. 6,020,459.

The full-length DP-178 polypeptide is based upon 36 amino acid residues spanning residues 638 to 673 of the transmembrane protein gp41 from the HIV-1_{LAI} isolate, and has an amino acid sequence:

YTSLIHSLIEESQNQQEKNEQELLELDKWASLWNWF.
(T-20; SEQ ID NO:1)

An analog of a DP-178 polypeptide used in this invention contains one or more amino acid substitutions (conserved or non-conserved), insertions and/or deletions of the full-length DP-178 polypeptide. Homologs of DP-178 from other virus isolates or species, or from other organisms, are also analogs of DP-178. In addition to the full-length DP-178 polypeptide (SEQ ID NO:1), a DP-178 polypeptide used in this invention includes truncations of the full-length DP-178 comprising 3 to 35 contiguous amino acid residues. DP-178 analogs can also be truncated. Truncations and analogs of DP-178 are described in U.S. Pat. No. 5,464,933, U.S. Pat. No. 6,020,459, and WO 96/40191.

An amino acid substitution in a DP-178 polypeptide used in this invention can be a conserved or non-conserved substitution. A conserved amino acid substitution replaces one or more amino acids of a DP-178 polypeptide sequence with amino acids of similar charge, size, and/or hydrophobicity characteristics, such as, for example, a glutamic acid (E) to aspartic acid (D). A non-conserved substitution replaces one or more amino acids of a DP-178 polypeptide sequence with amino acids possessing dissimilar charge, size, and/or hydrophobicity characteristics, such as, for example, a glutamic acid (E) to valine (V).

HIV-1 and HIV-2 enveloped proteins are structurally distinct, but there exists a striking amino acid conservation within the DP178-corresponding regions of HIV-1 and HIV-2. The amino acid conservation is of a periodic nature, suggesting some conservation of structure and/or function. Therefore, one class of amino acid substitutions includes amino acid changes which are predicted to stabilize the structure of DP-178 peptides of the invention. Utilizing the DP-178 full-length and DP-178 analog sequences described herein, and in U.S. Pat. No. 6,020,459, the skilled artisan can readily compile DP-178 consensus sequences, and ascertain the conserved amino acid residues which would represent preferred amino acid substitutions.

An amino acid insertion in a DP-178 polypeptide used in this invention includes one or more of a single amino acid residue or a stretch of residues inserted into DP-178 polypeptide. The one or more insertions can be made at the amino or carboxy terminus of a DP-178 polypeptide or at an internal position of the polypeptide. Preferably, an internal insertion is from 2 to 15 amino acids in length. Preferably, an amino or carboxy terminus insertion is about 2 to about 50 amino acids and corresponds to a polypeptide sequence of gp41 that is amino or carboxy to the full-length DP-178 amino acid sequence.

A DP-178 polypeptide used in this invention can contain one or more deletions. A deletion in a DP-178 polypeptide can be the removal of one or more single amino acid residues or one or more of a stretch of contiguous amino acids, and combinations thereof. A DP-178 polypeptide containing a deletion is at least 4 to 6 amino acids.

A DP-178 polypeptide homolog used in this invention is a full-length or truncated DP-178 from a source other than HIV-1_{LAI}, e.g., other HIV-1 and HIV-2 isolates, non-HIV-1_{LAI} enveloped viruses, non-enveloped viruses and non-viral organisms. A DP-178 homolog can also contain one or more deletions, insertions or substitutions, as described above. A DP-178 homolog can comprise, e.g., peptide sequences present in transmembrane proteins of enveloped viruses or polypeptide sequences present in non-enveloped and non-viral organisms. Homologs of DP-178 are described in U.S. Pat. No. 5,464,933, U.S. Pat. No. 6,020,459, and WO 96/40191, and include:

YTNTIYTLLEESQNQQEKNEQELLELDKWASLWNWF;
(DP-185; SEQ ID NO:3)
YTGIIYNLLEESQNQQEKNEQELLELDKWANLWNWF;
(SEQ ID NO:4)
YTSLIYSLLEKSQIQQEKNEQELLELDKWASLWNWF;
(SEQ ID NO:5) and
LEANISQSLEQAQIQQEKNMYELQKLNSWDVFTNWL,
(SEQ ID NO:6)

which are derived from HIV-1_{SF2}, HIV-1_{RF}, HIV-1_{MN}, and HIV-2_{NIHZ} isolates, respectively.

A DP-178 polypeptide used in this invention can have a modification to the amino and/or carboxy terminus to provide, e.g., increased bioavailability, stability, or reduced host immune recognition. For example, the terminal NH₂ group can be replaced by a hydrophobic group, carbobenzyl, dansyl, or t-butoxycarbonyl, an acetyl group, or a 9-fluorenylmethoxy-carbonyl (FMOC) group. A macromolecular group, e.g., a lipid-fatty acid conjugate, polyethylene glycol, carbohydrate or peptide group, can be covalently attached to the NH₂ or to the modification. The terminal COOH group can be replaced, e.g., by an amido group, a t-butoxycarbonyl group, or a covalently attached macromolecular group, e.g., a lipid-fatty acid conjugate, polyethylene glycol, carbohydrate or an additional polypeptide group. A macromolecular group can be attached to the COOH or the modification.

The following Table provides examples of DP-178 polypeptides that can be used in the present invention:

TABLE 3

| | |
|---|---|
| YTSLIHSLIEESQNQQEKNEQELLELDKWASLWNWF; | (SEQ ID NO:1) |
| YTNTIYTLLEESQNQQEKNEQELLELDKWASLWNWF; | (DP-185; SEQ ID NO:3) |
| YTGIIYNLLEESQNQQEKNEQELLELDKWANLWNWF; | (SEQ ID NO:4) |
| YTSLIYSLLEKSQIQQEKNEQELLELDKWASLWNWF; | (SEQ ID NO:5) |
| LEANISQSLEQAQIQQEKNMYELQKLNSWDVFTNWL; | (SEQ ID NO:6) |
| CGGNNLLRAIEAQQHLLQLTVWGIKQLQARILAVERYLKDQ; | (SEQ ID NO:7) |
| WMEWDREINNYTSLIGSLIEESQNQQEKNEQELLE; | (SEQ ID NO:8) |
| YTSLIHSLIEESQNQQEKNEQELLELDKWASLWNWFNITNWLWLIKFI; | (SEQ ID NO:9) |
| YTSLIHSLIEESQNQQEKNEQELLELDKWASLWNAF; | (SEQ ID NO:10) |
| YTSLIHSLIEESQNQQEKNEQELLELDKWASLANWF; | (SEQ ID NO:11) |
| YTSLIHSLIEESQNQQEKNEQQLLELDKWASLWNWF; | (SEQ ID NO:12) |
| YTSLIHSLIEESQNQQEKNEQELLQLDKWASLWNWF; | (SEQ ID NO:13) |
| YTSLIHSLIEESQNQQEKNQQELLQLDKWASLWNWF; | (SEQ ID NO:14) |
| YTSLIHSLQEESQNQQEKNEQELLELDKWASLWNWF; | (SEQ ID NO:15) |
| YTSLIHSLIEESQNQQEKNEQELLELDKWASLWNW; | (SEQ ID NO:16) |
| YTSLIHSLIEQSQNQQEKNEQELLELDKWASLWNWF; | (SEQ ID NO:17) |
| YTSLIHSLIQESQNQQEKNEQELLELDKWASLWNWF; | (SEQ ID NO:18) |
| YTSLIHSLIEESQNQQEKNEQQLLELDKWASLWNWF; | (SEQ ID NO:19) |
| YTSLIQSLIEESQNQQEKNEQQLLELDKWASLWNWF; | (SEQ ID NO:20) |
| YTSLIHSLIEESQNQQEKNEQELLELDKWASLFNFF; | (SEQ ID NO:21) |
| YTSLIHSLIEESQNLQEKNEQELLELDKWASLWNWF; | (SEQ ID NO:22) |
| YTSLIHSLIEESQNQQEKLEQELLELDKWASLWNWF; | (SEQ ID NO:23) |
| YTSLIHSLIEESQNQQEKNEQELLEFDKWASLWNWF; | (SEQ ID NO:24) |
| YTSLIHSLIEESQNQQEKNEQELLELDKWASPWNWF; | (SEQ ID NO:25) |
| YTSLIHSLIEESQNQQEKNEQELLELDKWASLWNSF; | (SEQ ID NO:26) |
| NKSLEQIWNNMTWMEWDREINNYTSLIHSLIEESQNQQEKNEQELLELDKASLWNWF; | (SEQ ID NO:27) |
| SLEQIWNNMTWMEWDREINNYTSLIHSLIEESQNQQ; | (SEQ ID NO:28) |
| LEQIWNNMTWMEWDREINNYTSLIHSLIEESQNQQE; | (SEQ ID NO:29) |
| EQIWNNMTWMEWDREINNYTSLIHSLIEESQNQQEK; | (SEQ ID NO:30) |
| QIWNNMTWMEWDREINNYTSLIHSLIEESQNQQEKN; | (SEQ ID NO:31) |
| IWNNMTWMEWDREINNYTSLIHSLIEESQNQQEKNE; | (SEQ ID NO:32) |
| WNNMTWMEWDREINNYTSLIHSLIEESQNQQEKNEQ; | (SEQ ID NO:33) |
| NNMTWMEWDREINNYTSLIHSLIEESQNQQEKNEQE; | (SEQ ID NO:34) |
| NMTWMEWDREINNYTSLIHSLIEESQNQQEKNEQEL; | (SEQ ID NO:35) |
| MTWMEWDREINNYTSLIHSLIEESQNQQEKNEQELLELDKWASLWNW; | (SEQ ID NO:36) |
| TWMEWDREINNYTSLIHSLIEESQNQQEKNEQELLE; | (SEQ ID NO:37) |
| WMEWDREINNYTSLIHSLIEESQNQQEKNEQELLEL; | (SEQ ID NO:38) |
| WMEWDREINNYTSLIHSLIEESQNQQEKNEQELLE; | (SEQ ID NO:39) |
| MEWDREINNYTSLIHSLIEESQNQQEKNEQELLELD; | (SEQ ID NO:40) |
| EWDREINNYTSLIHSLIEESQNQQEKNEQELLELDK; | (SEQ ID NO:41) |
| WDREINNYTSLIHSLIEESQNQQEKNEQELLELDKW; | (SEQ ID NO:42) |
| DREINNYTSLIHSLIEESQNQQEKNEQELLELDKWA; | (SEQ ID NO:43) |
| REINNYTSLIHSLIEESQNQQEKNEQELLELDKWAS; | (SEQ ID NO:44) |
| EINNYTSLIHSLIEESQNQQEKNEQELLELDKWASL; | (SEQ ID NO:45) |
| INNYTSLIHSLIEESQNQQEKNEQELLELDKWASLW; | (SEQ ID NO:46) |
| NYTSLIHSLIEESQNQQEKNEQELLELDKWASLWNW; | (SEQ ID NO:47) |
| TSLIHSLIEESQNQQEKNEQELLELDKWASLWNWFN; | (SEQ ID NO:48) |
| SLIHSLIEESQNQQEKNEQELLELDKWASLWNWFNI; | (SEQ ID NO:49) |
| LIHSLIEESQNQQEKNEQELLELDKWASLWNWFNIT; | (SEQ ID NO:50) |
| TSLIHSLIEESQNQQEKNEQELLELDKWASLWNWF; | (SEQ ID NO:51) |
| LIHSLIEESQNQQEKNEQELLELDKWASLWNWF; | (SEQ ID NO:52) |
| ESQNQQEKNEQELLELDKWASLWNWF; | (SEQ ID NO:53) |
| NQQEKNEQELLELDKWASLWNWF; | (SEQ ID NO:54) |
| EKNEQELLELDKWASLWNWF; | (SEQ ID NO:55) |
| LRAIEAQQHLLQLTVWQIKQLQARILAV; | (SEQ ID NO:56) |
| YTSLIYSLLEKSQIQQEKNEQELLELDKWASLWNWF; | (SEQ ID NO:57) |
| NNLLRAIEAQQGLLQLTVWGIKQLQARILAVERYLKDQ; | (SEQ ID NO:58) |
| NNLLRAIEAQQHLLQLTVWGIKQLQARILAVERYLKDQGGC; | (SEQ ID NO:59) |
| NNLLRAIEAQQHLLQLTVWGIKQLQARILAVERYLKDQGGC; | (SEQ ID NO:60) |
| CGGNNLLRAIEAQQHLLQLTVWGIKQLQARILAVERYKDQGGC; | (SEQ ID NO:61) |
| and | |
| LSGIVQQQNNLLRAIEAQQHLLQLTVWGIKQLQARILAV. | (SEQ ID NO:62) |

### DP-107 Peptides

Optionally, a DP-178 polypeptide can be used with a DP-107 polypeptide, which is described in U.S. Pat. No. 6,020,459 and in WO 96/40191. Since DP-107 and DP-178 portions of the transmembrane protein gp41 form non-covalent protein-protein interactions which is necessary for normal viral infectivity, the combination of a DP-178 and DP-107 peptides may form a complex with viral gp41 and interfere with the fusogenic process. Thus, it may be desirable to use a DP-107 polypeptide in a combination therapy of this invention.

A full-length DP-107 polypeptide is 38 amino acids corresponding to residues 558 to 595 of HIV-1_{LAI} transmembrane gp41 protein, as shown:

NNLLRAIEAQQHLLQLTVWQIKQLQARILAVERYLKDQ.
(SEQ ID NO:2)

A DP-107 polypeptide used in this invention includes truncations of the full-length DP-107 polypeptide, and contains from 3 to 38 contiguous amino acid residues. Examples of DP-107 truncations are described in U.S. Pat. No. 6,020,459 and WO 96/40191.

A DP-107 polypeptide used in this invention includes analogs of the full-length and of truncated DP-107, which analogs contain one or more amino acid substitutions (conserved or non-conserved), insertions and/or deletions, as described above for DP-178. Analogs of DP-107 are described in U.S. Pat. No. 6,020,459 and WO 96/40191. A DP-107 polypeptide can also contain a modification at the amino or carboxy terminus to provide, e.g., increased bioavailability, stability, or reduced host immune recognition, as described above for DP-178 and in U.S. Pat. No. 6,020,459 and WO 96/40191. Homologs of DP-107 from other virus isolates or species, or from other organisms, are also analogs of DP-107, and are described in U.S. Pat. No. 6,020,459 and WO 96/40191.

A DP-107 polypeptide used in this invention, whether full-length, truncated, an analog (i.e., containing an insertion, deletion, substitution, or a homolog of DP-107), or otherwise modified, exhibits antiviral activity, antifusogenic activity, or modulates intracellular processes involving coil-coil peptides, and has structural and/or amino acid motif similarity to DP-107, and can be identified or recognized by the ALLMOTI5, 107X178X4 and PLZIP search motifs described in detail in U.S. Pat. No. 6,020,459.

Preparation of Polypeptides

A polypeptide used in this invention can be synthesized or prepared by techniques well known in the art. See, e.g., Creighton, 1983, Proteins: Structures and Molecular Principles, W.H. Freeman and Co., NY. Short peptides, for example, can be synthesized on a solid support or in solution. Longer peptides can be made using recombinant DNA techniques. The nucleotide sequences encoding the peptides of the invention may be synthesized, and/or cloned, and expressed according to techniques well known in the art. See, e.g., Sambrook, et al., 1989, Molecular Cloning, A Laboratory Manual, Vols. 1-3, Cold Spring Harbor Press, NY.

A polypeptide used in this invention can be synthesized in a manner such that one or more of the bonds which link the amino acid residues of the peptides are non-peptide bonds, as described supra. A non-peptide bond can be formed by conventional reactions well known in the art, e.g., by imino, ester, hydrazide, semicarbazide, and azo bonds. Peptides used in the invention can also be synthesized having additional chemical groups (i.e., modifications, as described supra) present at the amino and/or carboxy termini, e.g., to enhance the stability, bioavailability, and/or inhibitory activity of the polypeptide according to conventional methodologies.

A polypeptide used in this invention can also be synthesized with an altered steric configuration, e.g., the D-isomer of one or more of the amino acid residues of the polypeptide is used versus the L-isomer. If desired, one or more amino acid residues of a polypeptide used in this invention can be substituted by a non-naturally occurring amino acid residue. Alterations such as these may serve to increase the stability, bioavailability and/or inhibitory action of the peptides of the invention.

Specific methods for synthesizing a DP-178 or DP-107 polypeptide used in this invention are described in U.S. Pat. No. 6,015,881, which use solid and liquid phase synthesis procedures to synthesize and combine groups of specific polypeptide fragments to yield the peptide of interest. Generally, these methods involve synthesizing specific side-chain protected peptide fragment intermediates of a peptide on a solid support, coupling the protected fragments in solution to form a protected peptide, followed by deprotection of the side chains to yield the final polypeptide.

Use of Additional Therapeutic Agents

A combination therapy of the present invention can further include one or more, preferably one to three, additional antiviral agents useful in anti-HIV therapy. Antivirals which act on a different target molecule involved in viral replication, which act on a different target molecule involved in viral transmission, which prevent or reduce the occurrence of viral resistance, and which act on a different loci of the same molecule, can be used with a combination therapy of the invention. Such classes of agents include those which cause an inhibition of viral reverse transcriptase, inhibition of viral mRNA capping, inhibition of the HIV protease, inhibition of protein glycosylation, inhibition of viral fusion (e.g., DP-107 as described above), and other antiviral drugs, some of which are discussed herein, not falling within the aforementioned acitivities. One skilled in the art can determine a wide variety of antiviral therapies using an antiviral agent that exhibits one of these modes of activity. In particular, the combinations known as HAART can be used with a combination of this invention. Preferably, lower doses of antiviral agents are used in a combination therapy of the invention, thus reducing the toxicity associated with that agent, without loss of antiviral activity and while reducing or avoiding viral resistance.

The term “nucleoside and nucleotide reverse transcriptase inhibitors” (“NRTI”s) as used herein means nucleosides and nucleotides and analogues thereof that inhibit the activity of HIV-1 reverse transcriptase, which catalyzes the conversion of viral genomic HIV-1 RNA into proviral HIV-1 DNA. Nucleoside derivatives include but are not limited to 2′,3′-dideoxyadenosine (ddA); 2′,3′-dideoxyguanosine (ddG); 2′,3′-dideoxyinosine (ddI); 2′,3′-dideoxycytidine (ddC); 2′,3′-dideoxythymidine (ddT); 2′,3′-dideoxy-dideoxythymidine (d4T) and 3′-azido-2′,3′-dideoxythymidine (AZT).

Alternatively, halogenated nucleoside derivatives can be used, preferably 2′,3′-dideoxy-2′-fluoronucleosides, e.g., 2′,3′-dideoxy-2′-fluoroadenosine; 2′,3′-dideoxy-2′-fluoroinosine; 2′,3′-dideoxy-2′-fluorothymidine; 2′,3′-dideoxy-2′-fluorocytosine; and 2′,3′-dideoxy-2′,3′-didehydro-2′-fluoronucleosides including, but not limited to 2′,3′-dideoxy-2′,3′-didehydro-2′-fluorothymidine (Fd4T). Preferably, the 2′,3′-dideoxy-2′-fluoronucleosides of the invention are those in which the fluorine linkage is in the beta configuration, e.g., 2′,3′-dideoxy-2′-beta-fluoroadenosine (F-ddA), 2′,3′-dideoxy-2′-beta-fluoroinosine (F-ddI), and 2′,3′-dideoxy-2′-beta-fluorocytosine (F-ddC).

Typical suitable NRTIs include zidovudine (AZT) available under the RETROVIR tradename from Glaxo-Wellcome Inc., (Research Triangle, N.C.); didanosine (ddI) available under the VIDEX tradename from Bristol-Myers Squibb Co. (Princeton, N.J.); zalcitabine (ddC) available under the HIVID tradename from Roche Pharmaceuticals, (Nutley, N.J.); stavudine (d4T) available under the ZERIT trademark from Bristol-Myers Squibb Co.; lamivudine (3TC) available under the EPIVIR tradename from Glaxo-Wellcome; abacavir (1592U89) disclosed in WO96/30025 and available under the ZIAGEN trademark; adefovir dipivoxil [bis(POM)-PMEA] available under the PREVON tradename from Gilead Sciences (Foster City, Calif.); lobucavir (BMS-180194), a nucleoside reverse transcriptase inhibitor disclosed in EP-0358154 and EP-0736533 and under development by Bristol-Myers Squibb, (Princeton, N.J.); BCH-10652, a reverse transcriptase inhibitor (in the form of a racemic mixture of BCH-10618 and BCH-10619) under development by Biochem Pharma, (Laval, Quebec, Canada); emitricitabine [(−)-FTC] licensed from Emory University's U.S. Pat. No. 5,814,639 and under development by Triangle Pharmaceuticals, (Durham, N.C.); beta-L-FD4 (also called beta-L-D4C and named beta-L-2′,3′-dicleoxy-5-fluoro-cytidene) licensed by Yale University to Vion Pharmaceuticals, (New Haven Conn.); DAPD, the purine nucleoside, (−)-beta-D-2,6,-diamino-purine dioxolane disclosed in EP 0656778 and licensed by Emory University and the University of Georgia to Triangle Pharmaceuticals; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc., (West Conshohoken, Pa.).

The term “non-nucleoside reverse transcriptase inhibitors” (“NNRTI”s) as used herein means non-nucleosides that inhibit the activity of HIV-1 reverse transcriptase. Typical suitable NNRTIs include nevirapine (BI-RG-587) available under the VIRAMUNE tradename from Boehringer Ingelheim, the manufacturer for Roxane Laboratories (Columbus Ohio); delaviradine (BHAP, U-90152) available under the RESCRIPTOR tradename from Pharmacia & Upjohn Co. (Bridgewater N.J.); efavirenz (DMP-266) a benzoxazin-2-one disclosed in WO94/03440 and available under the SUSTIVA tradename from DuPont Pharmaceutical Co. (Wilmington Del.); PNU-142721, a furopyridine-thio-pyrimide under development by Pharmacia & Upjohn Co.; AG-1549 (formerly Shionogi # S-1153); 5-(3,5-dichlorophenyl)-thio-4-isopropyl-1-(4-pyridyl)methyl-IH-imidazol-2-ylmethyl carbonate disclosed in WO 96/10019 and under clinical development by Agouron Pharmaceuticals, Inc. (LaJolla Calif.); MKC-442 (1-(ethoxy-methyl)-5-(1-methylethyl)-6-(phenylmethyl)-(2,4(1H,3H)-pyrimidinedione) discovered by Mitsubishi Chemical Co. and under development by Triangle Pharmaceuticals; and (+)-calanolide A (NSC-675451) and B, coumarin derivatives disclosed in U.S. Pat. No. 5,489,697 licensed to Med Chem Research, which is co-developing (+) calanolide A with Vita-Invest as an orally administrated product.

The term “protease inhibitor” describes an antiviral agent that inhibits the HIV-1 protease, which is required for the proteolytic cleavage of viral polyprotein precursors (e.g., viral GAG and GAG Pol polyproteins), into individual functional proteins found in infectious HIV-1. PIs may work primarily during or after virus assembly (i.e., viral budding) to inhibit the maturation of virions into a mature infectious state. HIV protease inhibitors include compounds having a peptidomimetic structure, high molecular weight (7600 daltons) and substantial peptide character, e.g., CRIXIVAN (available from Merck) as well as nonpeptide protease inhibitors, e.g., VIRACEPT (available from Agouron).

Typical suitable PIs include saquinavir (Ro 31-8959) available in hard gel capsules under the INVIRASE tradename and as soft gel capsules under the FORTOVASE tradename from Roche Pharmaceuticals; ritonavir (ABT-538) available under the NORVIR tradename from Abbott Laboratories (Abbott Park, Ill.); indinavir (MK-639) available under the CRIXIVAN tradename from Merck & Co., Inc., (West Point, Pa.); nelfnavir (AG-1343) available under the VIRACEPT tradename from Agouron Pharmaceuticals, Inc.; amprenavir (141W94), tradename AGENERASE, a non-peptide protease inhibitor under development by Vertex Pharmaceuticals, Inc., (Cambridge, Mass.) and available from Glaxo-Wellcome under an expanded access program; lasinavir (BMS-234475) available from Bristol-Myers Squibb (discovered by Novartis (CGP-61755); DMP450, a cyclic urea discovered by Dupont and under development by Triangle Pharmaceuticals; BMS-2322623, an azapeptide under development by Bristol-Myers Squibb, as a second generation HIV-1 PI; ABT-378 under development by Abbott; and AG-1549, an orally active imidazole carbamate discovered by Shionogi (Shionogi #S-1153) and under development by Agouron Pharmaceuticals, Inc.

A combination therapy of the present invention can be used in further combination with uridine phosphorylase inhibitors, e.g., acyclouridine compounds, including benzylacyclouridine (BAU), benzyloxybenzylacyclouridine (BBAU), aminomethylbenzylacyclouridine (AMBAU), aminomethylbenzyloxybenzylacyclouridine (AMB-BAU), hydroxymethylbenzylacyclouridine (HMBAU), and hydroxymethylbenzyloxybenzylacyclouridine (HMBBAU); cytokines or cytokine inhibitors, e.g., IL-2 [PROLEUKIN (aldesleukin), Chiron Corp. (Emeryville, Calif.) described in EP-0142268, EP-0176299, RE 33653, U.S. Pat. No. 4,530,787, U.S. Pat. No. 4,569,790, U.S. Pat. No. 4,604,377, U.S. Pat. No. 4,748,234, U.S. Pat. No. 4,752,585, and U.S. Pat. No. 4,949,314], IL-12 (disclosed in WO96/25171, Roche Pharmaceuticals and American Home Prodocts, Madison, N.J.), IFN-α (INTRON-A; Schering Plough), IFN-β and IFN-γ, and recombinant forms thereof, TNF-α inhibitors, and MNX-160; viral capping drugs which interfere with 5′-mRNA processing, e.g., Ribavirin (1-β-D-ribofuranosyl-1H-1,2,4-triazole-3-carboxamide; described in U.S. Pat. No. 4,211,771 ICN Pharmaceuticals, Inc., Costa Mesa, Calif.;); antiviral agents active against a variety of lipid-enveloped viruses including HIV, e.g., Amphotericin B or its methyl ester (Fungizone; Gibco) as a lipid binding molecule with antiviral (anti-HIV) activity; inhibitors of glycoprotein processing, e.g., castonospermine (Boehringer Mannheim); hydroxyurea (Droxia; Bristol-Myers Squibb), which is an inhibitor of ribonucleoside triphosphate reductase, shown in preclinical studies to have a synergistic effect on the activity of didanosine, and can be used with stavudine; Yissum Project No. 11607, which is a synthetic protein based on the HIV-1 Vif protein, under preclinical development by Yissum Research Development Co. (Jerusalem, Israel).

Antifungal agents and other agents against opportunistic infections, including TB, HBV, EBV, and CMV, can be used with combination therapy of the present invention for alleviating or treating a disease associated with HIV-infected, immunosuppressed patients

Preferred additional therapeutic agents used in a combination therapy of this invention include ddI, DP-107, 3TC, ribavirin, and IFN-β.

Antiviral Activity Assays

In vitro assays for the study of antiviral compounds active at different stages of HIV infection (acute, co-cultivation, and chronic) are well known in the art, e.g., see Lambert et al., Antiviral Res. 21: 327-342, (1993). These assays can be used to assess the effects of a combination therapy of this invention with additional therapeutic agents if desired. The antiviral activity exhibited by antiviral agents used in a combination therapy of this invention can be measured using in vitro assays, e.g., as described below, which test a DP-178 polypeptide's ability to inhibit syncytia formation or inhibit infection by cell-free virus, and/or test the CCR5 inhibitory and antagonistic activity of a CCR5 antagonist compound used in this invention. Using these assays, such parameters as the relative antiviral activity of the peptides, exhibit against a given strain of virus and/or the strain specific inhibitory activity of the peptide can be determined.

Furthermore, the effectiveness of the present combination therapy can be determined by the multiple drug analysis method of Chou and Talalay (Chou and Talalay, 1984, Adv. Enzyme Regul. 22:27-55) and ‘Dose-Effect Analysis with Microcomputer Software (Chou and Chou, 1987, Software and Manual. p. 19-64. Elsevier Biosoft, Cambridge, UK). Analysis of drug-drug interactions, including synergy and antagonism between antiviral agnets used in a combination therapy of this invention, can be calculated by the MacSynergy computer program (Pritchard and Shipman, 1990, Antiviral Research 14: 181-206).

CCR5 antagonist compounds of the invention inhibit RANTES (“regulated upon activation, normal T cell expressed secreted”, which is a natural ligand of CCR5) binding with activity (Ki) from about 0.1 to 2000 nM, preferably from 0.1 to 1000 nM, more preferably from about 0.1 to 500 nM, and most preferably from about 0.1 to 100 nM.

### CCR5 Membrane Binding Assay

A high throughput screen utilizing a CCR5 membrane binding assay identifies inhibitors of RANTES binding. This assay utilizes membranes prepared from NIH 3T3 cells expressing the human CCR5 chemokine receptor which have the ability to bind to RANTES. Using a 96-well plate format, membrane preparations are incubated with ¹²⁵I-RANTES in the presence or absence of compound for one hour. Compounds are serially diluted over a wide range of 0.001 μg/ml to 1 μg/ml and tested in triplicates. Reaction cocktails are harvested through glass fiber filters, and washed thoroughly. Total counts for replicates are averaged and data reported as the concentration required to inhibit 50 percent of total ¹²⁵I-RANTES binding. Compounds with potent activity in the membrane binding assay are further characterized in secondary cell-based HIV-1 entry and replication assays.

### HIV-1 Entry Assay

Replication defective HIV-1 reporter virions are generated by cotransfection of a plasmid encoding the NL4-3 strain of HIV-1 (which has been modified by mutation of the envelope gene and introduction of a luciferase reporter plasmid) along with a plasmid encoding one of several HIV-1 envelope genes as described in Connor et al, Virology 206: 935-944 (1995). Following transfection of the two plasmids by calcium phosphate precipitation, the viral supernatants can be harvested on day 3 and a functional viral titer determined. These stocks are then used to infect U87 cells stably expressing CD4 and the chemokine receptor CCR5, which have been preincubated with or without test compound. Infections are carried out for 2 hours at 37° C., the cells washed and media replaced with fresh media containing a CCR5 antagonist compound. The cells are incubated for 3 days, lysed and luciferase activity determined. Results are reported as the concentration of compound required to inhibit 50% of the luciferase activity in the control cultures.

### HIV-1 Replication Assay

This assay uses primary peripheral blood mononuclear cells or the stable U87-CCR5 cell line to determine the effect of antiviral agents used in the present combination therapy to block infection of primary HIV-1 strains. The primary lymphocytes are purified from normal healthy donors and stimulated in vitro with PHA and IL-2 three days prior to infection. Using a 96-well plate format, cells are pretreated with drug for 1 hour at 37° C. and subsequently infected with an M-tropic HIV-1 isolate. Following infection, the cells are washed to remove residual inoculum and cultured in the presence of compound for 4 days. Culture supernatants are harvested and viral replication measured by determination of viral p24 antigen concentration.

### Calcium Flux Assay

Cells expressing the HIV coreceptor CCR5 are loaded with calcium sensitive dyes prior to addition of compound or the natural CCR5 ligand (RANTES). Compounds with agonist properties will induce a calcium flux signal in the cell, while CCR5 antagonists are identified as compounds which do not induce signaling by themselves but are capable of blocking signaling by the natural ligand RANTES.

### GTPγS Binding Assay (Secondary Membrane Binding Assay)

A GTPγS binding assay measures receptor activation by CCR5 ligands. This assay measures the binding of ³⁵S labeled-GTP to receptor coupled G-proteins, which occurs as a result of receptor activation by an appropriate ligand. In this assay, the CCR5 ligand RANTES is incubated with membranes from CCR5 expressing cells and binding to the receptor (or activation) is determined by assaying for bound ³⁵S label. The assay quantitatively determines if compounds exhibit agonist characteristics by inducing activation of the receptor or alternatively antagonist properties by measuring inhibition of RANTES binding in a competitive or non-competitive fashion.

### Chemotaxis Assay

The chemotaxis assay is a functional assay which characterizes the agonist vs. antagonist properties of the test CCR5 antagonist compounds. The assay measures the ability of a non-adherent murine cell line expressing human CCR5 (BaF-550) to migrate across a membrane in response to either test compounds or natural ligands (i.e., RANTES, MIP-1β). Cells migrate across the permeable membrane towards compounds with agonist activity. Compounds that are antagonists not only fail to induce chemotaxis, but are also capable of inhibiting cell migration in response to known CCR5 ligands.

### Cell Fusion Assay

Assays for detecting cell fusion events are well known in the art, and can be used to determine the ability of a DP-178 polypeptide and/or CCR5 antagonist to inhibit membrane fusion or viral-induced syncytial. Cell fusion assays are generally performed in vitro, by culturing cells which, in the absence of any treatment, would typically undergo an observable level of syncytial formation. For example, uninfected cells (e.g., CD4⁺ cells such as Molt or MEM cells) are incubated in the presence of cells chronically infected with a virus (e.g. HIV) that induces cell fusion or in the presence or absence of a polypeptide to be assayed. After incubation (e.g. 24 hours at 37° C.) the cell culture is examined microscopically for the presence of multinucleated giant cells, which are indicative of cell fusion and syncytial formation. Well known stains, such as crystal violet stain, can be used to facilitate the visualization of syncytial formation.

Pharmaceutical Compositions, Dosaging and Administration Schedules

Since the DP-178 site of action is at the surface of the virus, and prevents free virus from infecting host cells and cell to cell transmission of the virus, the use of DP-178 in combination with a CCR5 antagonist compound, which blocks the host cell receptor for HIV, provides different therapeutic targets having mechanisms of action that can provide an additive or synergistic effect. Preferably, a combination of the present invention is used at lower concentrations of the antiviral agents, resulting in decreased toxicity. Thus, a combination therapy of this invention may not only reduce the effective dose of a drug required for antiviral activity, thereby reducing its toxicity, but may also improve the absolute antiviral effect as a result of attacking the virus through multiple mechanisms. The combinations of the present invention also provide a means for circumventing or decreasing the chance of development of viral resistance.

Administration of a DP-178 polypeptide, or a pharmaceutically acceptable derivative thereof, and a CCR5 antagonist compound, or a pharmaceutically acceptable salt thereof, “in combination” includes procedures in which both agents are administered together as an admixture (single dosage form), and also procedures in which the two agents are administered separately but simultaneously, e.g., through separate intravenous lines or oral and intravenous administration.

Administration “in combination” further includes sequential administrations of a DP-178 polypeptide and CCR5 antagonist compound in separate administrations such that one of the drugs is given first, followed by the second, e.g., as in cycling therapy (i.e., the administration of a first antiviral compound for a period of time, followed by the administration of a second antiviral agent for a period of time and repeating this sequential administration to reduce the development of resistance to one of the compounds).

A pharmaceutical composition suitable for use in the present invention is formulated using a therapeutically effective amount of the antiviral agents to achieve their intended purpose with a suitable pharmacological carrier. For all such purposes, the exact formulation, route of administration and dosage can be chosen by the clinician in view of the patient's condition (see, e.g., Fingl et al., 1975, in “The Pharmacological Basis of Therapeutics”, Ch. 1).

Procedures for formulation and administration may be found in “Remington's Pharmaceutical Sciences”, 18th ed., 1990, Mack Publishing Co., Easton, Pa. Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Routes of administration include oral, rectal, transmucosal, intestinal, parenteral, intramuscular, subcutaneous, intramedullary, intrathecal, direct intraventricular, intravenous (i.e., injection or continuous infusion), intraperitoneal, intranasal, inhalation, intraocular, transdermal, topical, vaginal, and the like.

A pharmaceutical preparation used in a combination therapy of the present invention is preferably in a unit dosage form. In such form, the preparation is subdivided into suitably sized unit doses containing an appropriate amount of the antiviral agent. Effective dosages of the peptides of the invention to be administered may be determined through procedures well known to those in the art which address such parameters as biological half-life, bioavailability, and toxicity.

Dosage forms include but are not limited to tablets, dispersions, powders, suspensions, suppositories, solutions, emulsions, capsules, dragee cores, aerosols, implants, creams, patches, minipumps, and the like. Most preferably, administration of a DP-178 polypeptide is intravenous. Most preferably, administration of a CCR5 antagonist compound is oral.

An amount of a CCR5 antagonist, or derivative thereof, in a unit dose preparation, particularly for oral administration, can vary from about 10 mg to about 500 mg. The actual dosage of a CCR5 antagonist can be varied depending upon such factors as age, condition and size of the patient, severity of the symptoms being treated, and type of HIV isolate, which can be determined by one skilled in the art. The total daily dosage can be divided and administered in portions during a day of administration as needed. A typical recommended total daily dosage regimen can range from about 20 mg to about 800 mg, preferably 25 mg to 600 mg, more preferably about 50 mg to 400 mg, most preferably 100 mg to 200 mg per day of administration.

In the combination therapy of this invention, a therapeutically effective amount of a CCR5 antagonist, or derivative thereof, can be administered daily or less than daily, e.g., two to four times per week or alternatively every other day. The dosage can also be divided into two to four doses, or a single dose can be provided. Preferably, a therapeutically effective amount of a CCR5 antagonist, or derivative thereof, is administered orally each day, twice per day (bid). The practitioner may determine that the efficacy of the present combination allows less frequent administration of a CCR5 antagonist, e.g., once per day or less frequent, e.g., from 1 to 3 times per week or every other day.

An amount of a DP-178 polypeptide, or a pharmaceutically acceptable derivative thereof, is administered, in a unit dose preparation can be administered parenterally, e.g., as a continuous infusion or injection. Doses of a DP-178 polypeptide can range from 3 mg to 200 mg (or a multiple thereof to reduce viral load by 1 to 2 logs), preferably from 7 mg to 100 mg, more preferably from 20 mg to 35 mg, per day of administration. It is an advantage of the present invention that the polypeptide can be administered less often than twice per day (bid), which was previously reported for the use of T-20 in clinical trials. Thus, a combination of the present invention permits administration of a therapeutically effective amount of DP-178 polypeptide once per day (qid), preferably once (qd) two to four times per week, more preferably once (qd) every other day.

The doses and dosage regimens of the NRTIs, NNRTIs, Pls and other therapeutic agents used in combination with a therapy of this invention can be determined by the attending clinician in view of the approved doses and dosage regimens in the package inserts or as set forth in the protocols for the particular agent, taking into consideration the age, sex and condition of the patient, the severity of the condition treated and the HIV isolate.

Typical suitable multidrug combination therapies such as (i) at least three anti-HIV-1 drugs selected from two NRTIs, one PI, a second PI, and one NNRTI; and (ii) at least two antiviral agents selected from NNRTIs and PIs. Typical suitable HAART-multidrug combination therapies include:
(a) triple combination therapies such as two NRTIs and one PI; or
(b) two NRTIs and one NNRTI; and
(c) quadruple combination therapies such as two NRTIs, one PI and a second PI or one NNRTI.
   These multidrug combinations can be used in combination with a CCR5 antagonist and a DP-178 polypeptide, or their pharmaceutically acceptable salts/derivatives.

In treatment of naive patients, it is preferred to start anti-HIV-1 treatment with the triple multidrug combination therapy with a CCR5 antagonist and a DP-178 polypeptide, or their pharmaceutically acceptable salts/derivatives; the use of two NRTIs and one PI is prefered unless there is intolerance to PIs. Drug compliance is essential. The CD⁴⁺ and HIV-1-RNA plasma levels should be monitored every 3-6 months. Should viral load plateau, an additional drug, e.g., one PI or one NNRTI can be added. Typical therapies for use in combination with the present invention are further described in the following table:

ANTIVIRAL MULTI DRUG COMBINATION THERAPIES

A. Triple Combination Therapies
1. Two NRTIs¹ + one PI²
2. Two NRTIs¹ + one NNRTI³
B. Quadruple Combination Therapies⁴
Two NRTIs + one PI + a second PI or one NNRTI
C. Alternatives:⁵
Two NRTI¹
One NRTI⁵ + one PI²
Two PIs⁶ + one NRTI⁷ or NNRTI³
One PI² + one NRTI⁷ + one NNRTI³
Footnotes to Table
¹One of the following: zidovudine + lamivudine; zidovudine + didanosine; stavudine + lamivudine; stavudine + didanosine; zidovudine + zalcitabine.
²Indinavir, nelfinavir, ritonavir or saquinavir soft gel capsules.
³Nevirapine or delavirdine.
⁴See Vandamne et al., Antiviral Chemistry & Chemotherapy 9:193-197 (1998).
⁵Alternative regimens are for patients unable to take a recommended regimen because of compliance problems or toxicity, and for those who fail or relapse on a recommended regimen. Double nucleoside combinations may lead to HIV-resistance and clinical failure in many patients.
⁶Most data obtained with saquinavir and ritonavir (each 400 mg bid).
⁷Zidovudine, stavudine or didanosine.

1. A method of treating an HIV infection in an individual in need of such treatment, comprising administering in combination a therapeutically effective amount of a CCR5 antagonist, or a pharmaceutically acceptable salt thereof, and a therapeutically effective amount of a DP-178 polypeptide, or a pharmaceutically acceptable derivative thereof.

2. The method of claim 1, wherein the dosage of the CCR5 antagonist, or a pharmaceutically acceptable salt thereof, is 25 to 600 mg, and the dosage of the DP-178 polypeptide, or a pharmaceutically acceptable derivative thereof, is 3 to 200 mg, or a multiple thereof to reduce the viral load in the individual by 1 to 2 logs.

3. The method of claim 1, wherein the CCR5 antagonist, or a pharmaceutically acceptable salt thereof, is administered 1 or 2 times per day of administration.

4. The method of claim 1, wherein the CCR5 antagonist, or a pharmaceutically acceptable salt thereof, is administered from 1 to 3 times per week or every other day.

5. The method of claim 1, wherein the DP-178 polypeptide is administered 1 to 3 times per week, or every other day.

6. The method of claim 1, wherein the DP-178 polypeptide is administered 3 times per week or every other day.

7. The method of claim 1, wherein the CCR5 antagonist is administered orally.

8. The method of claim 1, wherein the DP-178 polypeptide is administered subcutaneously.

9. The method of claim 1, wherein the CCR5 antagonist is a compound selected from the group consisting of:

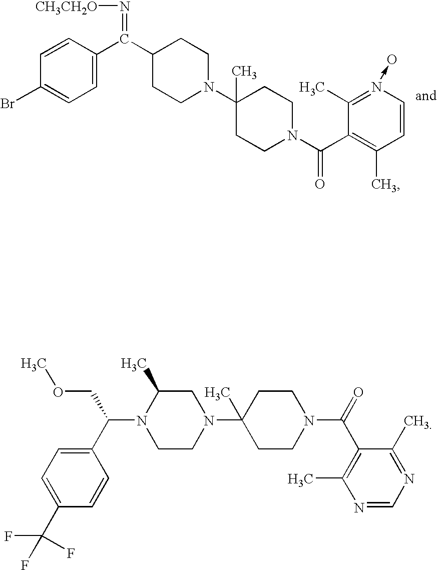

10. The method of claim 1, wherein the DP-178 polypeptide consists of an amino acid sequence in Table 3.

11. The method of claim 1, further comprising administering in combination one or more antiviral or therapeutic agents useful for the treatment of HIV.

12. The method of claim 11 wherein the antiviral agent is selected from the group consisting of nucleoside reverse transcriptase inhibitors, non-nucleoside reverse transcriptase inhibitors and protease inhibitors.

13. The method of claim 11 wherein the antiviral agent is selected from the group consisting of a DP-107 polypeptide, zidovudine, lamivudine, zalcitabine, didanosine, stavudine, abacavir, adefovir dipivoxil, lobucavir, BCH-10652, emitricitabine, beta-L-FD4, DAPD, lodenosine, nevirapine, delaviridine, efavirenz, PNU-142721, AG-1549, MKC442, (+)-calanolide A and B, saquinavir, indinavir, ritonavir, nelfinavir, lasinavir, DMP-450, BMS-2322623, ABT-378, amprenavir, hydroxyurea, ribavirin, IL-2, IL-12, Yissum No. 11607 and AG-1549.

14. A method of treating an HIV infection in an individual in need of such treatment, comprising orally administering from 25 to 400 mg/day, one to two times per day, of a CCR5 antagonist of structural formula 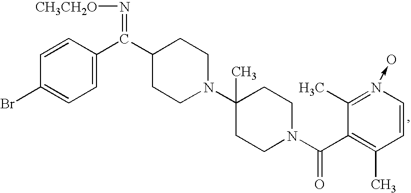
or a pharmaceutically acceptable salt thereof,
and subcutaneously administering from 3 to 200 mg, or a multiple thereof which reduces the viral load in the individual, once two, three or four times per week, or once every other day, of a T-20.

15. The method of claim 14, further comprising administering in combination one or more antiviral or therapeutic agents useful in the treatment of HIV.

16. The method of claim 15, wherein the antiviral agent is selected from the group consisting of nucleoside reverse transcriptase inhibitors, non-nucleoside reverse transcriptase inhibitors and protease inhibitors.

17. The method of claim 15, wherein the antiviral agent is selected from the group consisting of a DP-107 polypeptide, zidovudine, lamivudine, zalcitabine, didanosine, stavudine, abacavir, adefovir dipivoxil, lobucavir, BCH-10652, emitricitabine, beta-L-FD4, DAPD, lodenosine, nevirapine, delaviridine, efavirenz, PNU-142721, AG-1549, MKC442, (+)-calanolide A and B, saquinavir, indinavir, ritonavir, nelfinavir, lasinavir, DMP450, BMS-2322623, ABT-378, amprenavir, hydroxyurea, ribavirin, IL-2, IL-12, Yissum No. 11607 and AG-1549.

18. A kit comprising single package pharmaceutical compositions for use in combination to treat HIV infection, which comprises in a first container a pharmaceutical composition comprising a CCR5 antagonist, or pharmaceutically acceptable salt thereof, in a pharmaceutically acceptable carrier, in an oral dosage from 25 to 600 mg to be administered from 1 to 3 times per week or every other day, and in a second container a pharmaceutical composition comprising a DP-178 polypeptide, or a pharmaceutically acceptable derivative thereof, in a pharmaceutically acceptable carrier, in a subcutaneous dosage from 3 to 200 mg, or a multiple thereof which reduces the viral load by 1 or 2 logs.

19. The kit of claim 18 which comprises in additional container(s) one or more pharmaceutical compositions comprising a therapeutically effective amount of an antiviral or therepeutic agent useful in the treatment of HIV in a pharmaceutically acceptable carrier.
